(19) United States
(12) Patent Application Publication
(10) Pub. No.: US 2025/0367282 A1
(43) Pub. Date: Dec. 4, 2025

(54) POLYCATION CARRIER PARTICLE

(71) Applicant: ALLARTA LIFE SCIENCE INC., Dundas (CA)

(72) Inventors: Samantha ROS, Hamilton (CA); Mitchell Arbuthnot JOHNSON, Hamilton (CA); Nicole Aliida MANGIACOTTE, East Aurora, NY (US); Nicholas Alexander Despard BURKE, Dundas (CA); Harald Donald Helmut STOVER, Dundas (CA)

(21) Appl. No.: 18/871,946

(22) PCT Filed: Jun. 8, 2023
(86) PCT No.: PCT/CA2023/050785

§ 371 Date: Dec. 5, 2024

(30) Foreign Application Priority Data
Jun. 8, 2022 (US) 63350275

(51) Int. Cl. A61K 39/385; A61K 39/00

(52) U.S. Cl. CPC A61K 39/385; A61K 2039/53

(57) ABSTRACT

A polycation carrier particle obtained by the polymerization of a multiacrylate monomer with one or more hydrophilic monomers to form crosslinks. The polycation carrier particle has a net positive electric charge. The multiacrylate monomer and the hydrophilic monomer(s) are as defined herein.

### CROSS REFERENCE TO A RELATED APPLICATION

This application claims priority to U.S. provisional application No. 63/350,275 filed on Jun. 8, 2022, the contents of which are incorporated herein by reference.

### TECHNICAL FIELD

This disclosure relates to the field of carrier particles for therapeutic agents, vaccines, and the like, more particularly to polycation particles, as well as methods and uses thereof and the fabrication of same.

### BACKGROUND OF THE ART

In vivo delivery of proteins and genetic material in the form of polynucleic acids such as mRNA, DNA, and siRNA has the potential for a wide range of therapeutic application from gene knockdown and editing to mRNA/DNA and protein-based vaccines. The success of applying proteins and polynucleic acids for in vivo therapeutics is dependent on the ability to deliver the desired genetic cargo intracellular to targeted cells. This delivery of proteins and large polynucleic acids requires use of delivery vehicles to provide stability to the cargo during circulation and to promote cellular uptake often by endocytosis.

Many approaches based on synthetic materials have been used to produce the delivery vehicles. Most notably, lipid nanoparticles-based delivery of mRNA for COVID-19 vaccines developed by Pfizer and Moderna has achieved widespread clinical success. Polycations have also been explored as synthetic materials for both nucleic acid complexation and protein delivery. Proteins are protected by a polycation carrier particle prior to endocytosis and are released from the endosome by way of the proton sponge effect. The benefits of synthetic polycations over that of lipid nanoparticles include the ease of production, stability, low cost, as well the synthetic control over polymer architecture and composition that allows for tunability of features such as circulation time, release kinetics, and targeted cell delivery.

A significant challenge for the use of polycations in the delivery of nucleic acid materials for example is the associated general cytotoxicity of polycations. The dissociation of the complex between the polycation and the encapsulated material (e.g. a genetic payload) after cellular uptake results in the release of the encapsulated material as well as the potentially cytotoxic polycations. Accordingly, improvements are desired in polycation carrier particles in order to reduce the cytotoxicity of the polycations once the particles dissociate inside cells to release the encapsulated material.

### SUMMARY

In one aspect, there is provided a polycation carrier particle obtained by the polymerization of an acrylate monomer with one or more hydrophilic monomers to form crosslinks, wherein the polycation carrier particle has a net positive electric charge and wherein the acrylate monomer is selected from formula I or formula V.

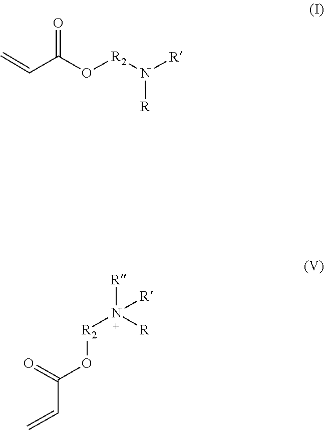

R and R″ are each independently a hydrogen, a C₁-C₁₄ linear or branched alkyl, a C₃-C₈ cycloalkyl, or a 5 to 10 membered aryl or heteroaryl ring, optionally terminated by a polymerizable group, preferably an acrylate group, capable of crosslinking with the one or more hydrophilic monomers. R′ is a C₂-C₁₄ linear or branched alkyl, a C₃-C₈ cycloalkyl, or a 5 to 10 membered aryl or heteroaryl ring, and R′ is terminated by at least one polymerizable group, preferably an acrylate group, capable of crosslinking with the one or more hydrophilic monomers. The alkyl, cycloalkyl, aryl and heteroaryl can be optionally substituted and optionally interrupted by one or more oxygen, sulfur or nitrogen atoms. And, R₂ is C₂ or C₃ and C₃ is linear or cyclic.

In some embodiments, the polymerizable group is selected from an acrylate group, a methacrylate group, a (meth)acrylamide group, or a styrene group.

In some embodiments, the acrylate monomer of formula I is a multiacrylate monomer of formula II

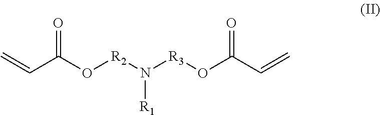

R₁ is hydrogen or a C₁-C₁₄ linear, branched or cyclic alkyl that is optionally substituted and optionally interrupted by one or more oxygen, sulfur or nitrogen atoms and optionally terminated with an acrylate group, R₂ is C₂ or C₃ and C₃ is linear or cyclic, and R₃ is a C₂-C₁₄ linear or branched alkyl, that is optionally substituted and optionally interrupted by one or more oxygen, sulfur or nitrogen atoms.

In some embodiments, the multiacrylate monomer is of formula III.

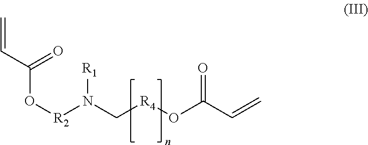

R₁, and R₂ are as previously defined, and R₄ is —CH₂—CH₂—O— or —CH₂—CH₂—CH₂—O— and the terminal CH₂ group of R₄ is linked to the oxide of the terminal acrylate group, and n is an integer selected from 1, 2, 3, or 4.

In some embodiments, the multiacrylate monomer is of formula IV.

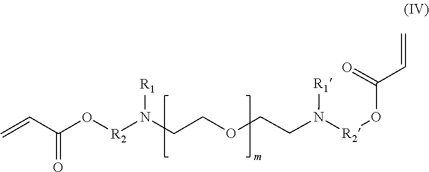

R₁ and R₂ are as previously defined, R₁′ has the same definition as R₁, R₂′ is C₂-C₃ (C₃ is linear or cyclic) and m is an integer selected from 0, 1, 2, 3 or 4. In some embodiments, one or more of the nitrogen atoms of formula IV are protonated.

In some embodiments, the acrylate monomer of formula V is a multiacrylate monomer of formula VI.

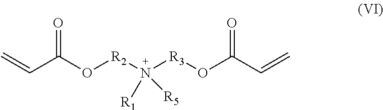

R₁ and R₅ are independently hydrogen or a C₁-C₁₄ linear, branched or cyclic alkyl that is optionally substituted and optionally interrupted by one or more oxygen, sulfur or nitrogen atoms and optionally terminated with an acrylate group, R₂ is C₂ or C₃ (C₃ is linear or cyclic), and R₃ is a C₂-C₁₄ linear or branched alkyl, that is optionally substituted and optionally interrupted by one or more oxygen, sulfur or nitrogen atoms.

In some embodiments, the multiacrylate monomer is of formula VII.

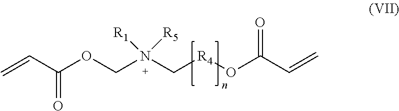

R₁, and R₅ are as previously defined, and R₄ is —CH₂—CH₂—O— or —CH₂—CH₂—CH₂—O— and the terminal CH₂ group of R₄ is linked to the oxide of the terminal acrylate group, and n is an integer selected from 1, 2, 3, or 4.

In some embodiments, the multiacrylate monomer is of formula VIII.

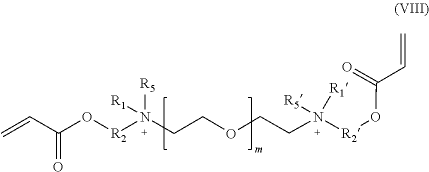

R₁ and R₅ are as previously defined, R₂ is C₂ or C₃ (C₃ is linear or cyclic), m is an integer selected from 0, 1, 2, 3, or 4 and R₁, R₂′ and R₅′ independently share the definitions of R₁, R₂ and R₅, respectively.

In some embodiments, the acrylate monomer is selected from:

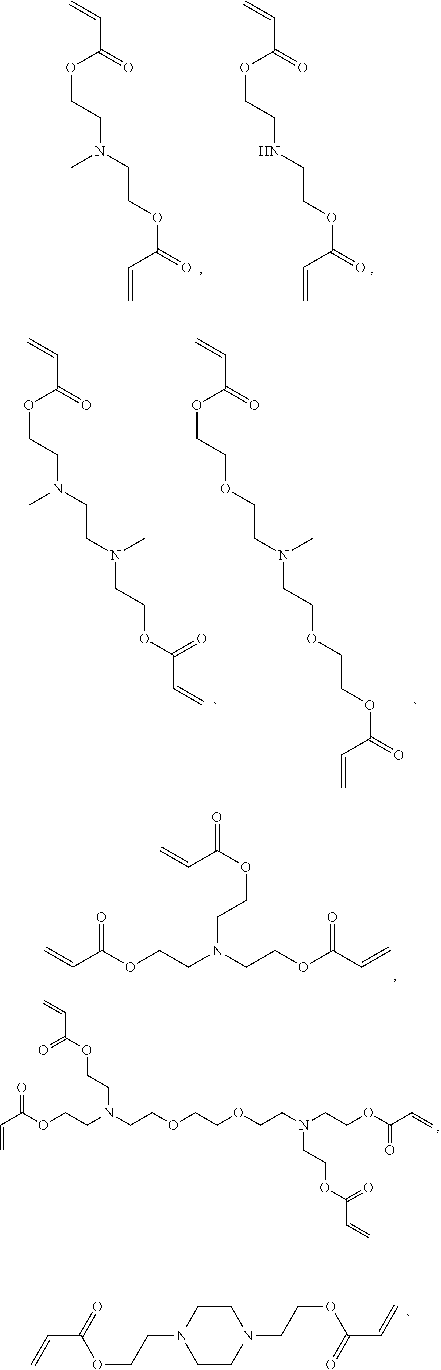

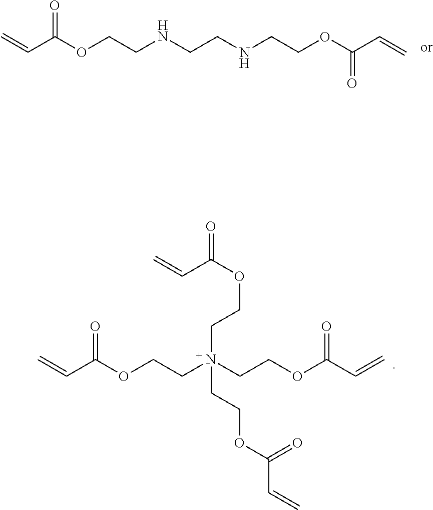

In some embodiments, the multiacrylate monomer is methyldiethanolamine diacrylate (DXL).

In some embodiments, the hydrophilic monomer is of formula IX.

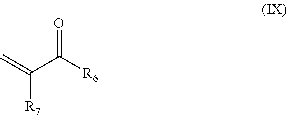

R₆ is —O—(CH₂)ₚ—Y, —NJK or —NJ₂K⁺, p is an integer selected from 2, 3, or 4, Y is defined as OH, COOH, NJ₂, NJ₃⁺, or ONJ₂ and each J is independently defined as H, methyl or ethyl, and K is ethyl, propyl or butyl that is optionally branched and optionally substituted, and R₇ is hydrogen or methyl.

In some embodiments, the hydrophilic monomer is of formula X.

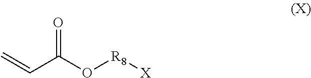

R₈ is a C₂-C₄ linear alkyl and X is selected from hydroxyl, carboxyl, tertiary amine, quaternary ammonium or amide, and the amine is optionally substituted by methyl or ethyl groups.

In some embodiments, the one or more hydrophilic monomers are 2-hydroxyethylacrylate (HEA) and/or N,N-(dimethylamino)ethyl acrylate (DMAEA).

In some embodiments, the polycation carrier particle has a size of from 200 nm to 5 μm.

In some embodiments, a molar percent ratio of the acrylate monomer to the one or more hydrophilic monomers is from 15/85 to 50/50.

In some embodiments, the polycation carrier particle further comprises a cargo material complexed with the polycation carrier particle.

In one aspect, there is provided a complex comprising polycation carrier particle complexed to a cargo molecule, the polycation carrier particle adapted to release the cargo molecule intracellularly and being degradable under physiological conditions, and wherein the polycation carrier particle has charge shifting properties and is obtained by polymerizing one or more hydrophilic monomers with an acrylate monomer of formula I or formula V as defined herein. In one embodiment, the cargo molecule is selected from a nucleic acid, a peptide and a protein.

In one aspect, there is provided a method of producing a polycation carrier particle as defined herein, the method comprising: providing an acrylate monomer as defined herein and one or more hydrophilic monomers as defined herein, and, crosslinking the acrylate monomer with the one or more hydrophilic monomers.

In some embodiments, the step of crosslinking comprises performing one of a precipitation polymerization, a micro emulsion polymerization, a dispersion polymerization, an inverse-suspension polymerization or an inverse emulsion polymerization.

In some embodiments, only one hydrophilic monomer is provided and the acrylate monomer to the hydrophilic monomer molar percent ratio is 30±15/70±15. In one example, the one hydrophilic monomer is HEA and the acrylate monomer is DXL.

In some embodiments, two hydrophilic monomers are provided and the acrylate monomer to the two hydrophilic monomers molar percent ratio is 33±10/33±10/33±10. For example, the two hydrophilic monomers are HEA and DMAEA and the acrylate monomer is DXL.

In some embodiments, the method further comprising providing an agent to be encapsulated and cross-linking the acrylate monomer with the one or more hydrophilic monomers in a medium comprising the agent.

In a further aspect, there is provided a method of producing a complex of a cargo material and a polycation carrier particle comprising: producing the polycation carrier particle by a method as defined herein; and complexing the cargo material having a negative charge to the polycation carrier particle having a positive charge by exposing the polycation carrier particle to a solution containing the cargo material, in order to obtain the complex.

In one aspect, there is provided a method of vaccine delivery to a subject in need thereof, the method comprising administering to the subject the polycation carrier particle as defined herein wherein the polycation carrier particle is complexed with a vaccine agent or encapsulates a vaccine composition.

In one aspect, there is provided a method of inducing an immune response in vivo comprising administering to a subject in need thereof the polycation carrier particle as defined herein complexed with an antigen or a nucleic acid encoding an antigen or encapsulating a vaccine composition.

In one aspect, there is provided a method of nucleic acid delivery for in vivo protein expression, comprising administering to a subject the polycation carrier particle as defined herein, where the polycation carrier particle is complexed with nucleic acid encoding the protein.

In one aspect, there is provided a vaccine composition comprising the polycation carrier particle as defined herein complexed with an antigen or a nucleic acid encoding an antigen and a pharmaceutically acceptable carrier or adjuvant. In one embodiment, the nucleic acid is mRNA.

Many further features and combinations thereof concerning the present improvements will appear to those skilled in the art following a reading of the instant disclosure.

### DESCRIPTION OF THE DRAWINGS

FIG. 1 is a ¹H nuclear magnetic resonance (NMR) spectrum of N-methyldiethanolamine diacrylate[(Methylazanediyl)bis(ethane-2,1-diyl)diacrylate] (DXL) in CDCl₃ recorded at 600 MHZ.

FIG. 2 shows a ¹H NMR spectra of DXL in 100 phosphate buffered D₂O at pH 7.30 and room temperature (22° C.) over time.

FIG. 3 is a graph showing the ester hydrolysis of DXL over time, more specifically the ester hydrolysis of DXL monomer in 100 mM phosphate buffered D₂O at pH 7.30, room temperature (22° C.). The percent (%) hydrolysis data represents hydrolysis of one of the ester groups of DXL.

FIG. 4 is a graph showing the pseudo first-order plot of ester hydrolysis of DXL monomer in 100 mM phosphate buffered D₂O at pH 7.30, room temperature (22° C.). The data represents hydrolysis of one of the ester groups of DXL.

FIG. 5 is a bright field microscopy image with 50× objective lens and a scale bar of 5 μm showing a polymerization mixture of a 70/30 molar feed ratio of 2-hydroxyethyl acrylate (HEA)/DXL in a 75/25 M methyl ethyl ketone (MEK)/heptane solvent mixture after 20-24 h of photopolymerization.

FIG. 6 is a bright field microscopy image with 50× objective lens and a scale bar of 5 μm showing a polymerization mixture of a 70/30 molar feed ratio of HEA/DXL in a 75/25 MEK/heptane solvent mixture after 5-8 h of thermal polymerization at 70° C., following purification and sonication in 1,4-dioxane.

FIG. 7 is a bright field microscopy image with 50× objective lens and a scale bar of 10 μm showing a suspension of a 1/1/1 molar feed ratio of DXL/N,N-(dimethylamino)ethyl acrylate (DMAEA)/HEA particles prepared in an 85/15 MEK/heptane solvent mixture thermal polymerization, following purification and sonication in 1,4-dioxane.

FIG. 8 is a bright field microscopy image with 50× objective lens and a scale bar of 5 μm showing a polymerization mixture of a 1/1/1 DXL/DMAEA/HEA particles in an 85/15 MEK/heptane solvent mixture following photopolymerization.

FIG. 9 is a bright field microscopy image with 50× objective lens and a scale bar of 10 μm showing a polymerization mixture of a 70/30 molar feed ratio of HEA/DXL in a 75/25 MEK/heptane solvent mixture after 20-24 h of photopolymerization, washed with 1,4-dioxane, and sonicated in phosphate buffered saline (PBS) at pH 7.4.

FIG. 10A is a confocal microscopy image of particles obtained with 70 mol % HEA and 30 mol % DXL in a 75/25 MEK/heptane solvent mixture at a 1 g total monomer loading. 1 mol % of azobisisobutyronitrile (AIBN) as a free-radical initiator for photo decomposition, washed twice with phosphate buffer saline, imaged on a confocal with 20× objective (the scale bars indicating 10 μm) and showing the merged channels: brightfield, fluorescein isothiocyanate (FITC), and tetramethyl rhodamine isothiocyanate (TRITC).

FIG. 10B is a confocal microscopy image of 70:30 HEA/DXL particles imaged with a 20× objective (the scale bars indicating 10 μm) and showing the brightfield channel.

FIG. 10C is a confocal microscopy image of 70:30 HEA/DXL particles imaged with a 20× objective (the scale bars indicating 10 μm) and showing the FITC channel.

FIG. 10D is a confocal microscopy image of 70:30 HEA/DXL particles imaged with a 20× objective (the scale bars indicating 10 μm) and showing the TRITC channel.

FIG. 11A is a confocal microscopy image of 70:30 HEA/DXL particles combined in a 1:1 wt ratio with ovalbumin Texas Red™ conjugate (OVA-RED), washed twice with PBS, and imaged on confocal with a 20× objective with the scale bars indicating 25 μm showing the brightfield, FITC, and TRITC channels merged.

FIG. 11B is a confocal microscopy image of 70:30 HEA/DXL particles combined in a 1:1 wt ratio with OVA-RED, washed twice with PBS, and imaged on confocal with 20× objective with the scale bars indicating 25 μm showing the brightfield channel.

FIG. 11C is a confocal microscopy image of 70:30 HEA/DXL particles combined in a 1:1 wt ratio with OVA-RED, washed twice with PBS, and imaged on confocal with 20× objective with the scale bars indicating 25 μm showing the FITC channel.

FIG. 11D is a confocal microscopy image of 70:30 HEA/DXL particles combined in a 1:1 wt ratio with OVA-RED, washed twice with PBS, and imaged on confocal with 20× objective with the scale bars indicating 25 μm showing the TRITC channel.

FIG. 12A is a confocal microscopy image of 70:30 HEA/DXL particles combined in a 1:1 wt ratio with OVA-RED, washed twice with PBS, imaged on confocal with 20× objective with the scale bars indicating 25 μm.

FIG. 12B is a confocal microscopy image of 70:30 HEA/DXL particles without OVA-RED, washed twice with PBS, imaged on confocal with 20× objective with the scale bars indicating 10 μm (B).

FIG. 13A is a brightfield microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free Dulbecco's modified Eagle medium (DMEM) supplemented with 1% penicillin-streptomycin. The cells were washed twice with PBS, fixed by 4% v/v paraformaldehyde in sterile water, stored in PBS, and imaged by brightfield microscopy with a 20× objective with the scale bars indicating 50 μm.

FIG. 13B is a brightfield microscopy image of RAW 264.7 cells in a glass bottom 96-well plate (Cellvis) after being incubated at 37° C. in a humidified atmosphere with 5% CO₂ for 4 h with 10% v/v dimethyl sulfoxide (DMSO) in serum-free DMEM. The cells were washed twice with PBS, fixed by 4% v/v paraformaldehyde in sterile water, stored in PBS, and imaged by brightfield microscopy with a 20× objective with the scale bars indicating 50 μm.

FIG. 13C is a brightfield microscopy image of RAW 264.7 cells in a glass bottom 96-well plate (Cellvis) after being incubated at 37° C. in a humidified atmosphere with 5% CO₂ for 4 h in serum-free DMEM. The cells were washed twice with PBS, fixed by 4% v/v paraformaldehyde in sterile water, stored in PBS, and imaged by brightfield microscopy with a 20× objective with the scale bars indicating 50 μm.

FIG. 14A is a confocal microscopy image of RAW 264.7 cells in a glass bottom 96-well plate (Cellvis) after being incubated at 37° C. in a humidified atmosphere with 5% CO₂ for 4 h with serum-free DMEM supplemented with 1% penicillin-streptomycin. The cells were washed twice with PBS, fixed by 4% v/v paraformaldehyde in sterile water and stored in PBS and imaged on confocal with 60× oil immersion objective with the scale bars indicating 10 μm, showing the merged brightfield, FITC, and TRITC channels.

FIG. 14B is a confocal microscopy image of RAW 264.7 cells in a glass bottom 96-well plate (Cellvis) after being incubated at 37° C. in a humidified atmosphere with 5% CO₂ for 4 h with serum-free DMEM supplemented with 1% penicillin-streptomycin. The cells were washed twice with PBS, fixed by 4% v/v paraformaldehyde in sterile water and stored in PBS and imaged on confocal with 60× oil immersion objective with the scale bars indicating 10 μm, showing the brightfield channel.

FIG. 14C is a confocal microscopy image of RAW 264.7 cells in a glass bottom 96-well plate (Cellvis) after being incubated at 37° C. in a humidified atmosphere with 5% CO₂ for 4 h with serum-free DMEM supplemented with 1% penicillin-streptomycin. The cells were washed twice with PBS, fixed by 4% v/v paraformaldehyde in sterile water and stored in PBS and imaged on confocal with 60× oil immersion objective with the scale bars indicating 10 μm, showing the FITC channel.

FIG. 14D is a confocal microscopy image of RAW 264.7 cells in a glass bottom 96-well plate (Cellvis) after being incubated at 37° C. in a humidified atmosphere with 5% CO₂ for 4 h with serum-free DMEM supplemented with 1% penicillin-streptomycin. The cells were washed twice with PBS, fixed by 4% v/v paraformaldehyde in sterile water and stored in PBS and imaged on confocal with 60× oil immersion objective with the scale bars indicating 10 μm, showing the TRITC channel.

FIG. 15A is a confocal microscopy image of RAW 264.7 cells in a glass bottom 96-well plate (Cellvis) after being incubated at 37° C. in a humidified atmosphere with 5% CO₂ for 4 h with 0.1 wt. % HD particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling, the scale bars indicates 10 μm and the merged brightfield, FITC, and TRITC channels are shown.

FIG. 15B is a confocal microscopy image of RAW 264.7 cells in a glass bottom 96-well plate (Cellvis) after being incubated at 37° C. in a humidified atmosphere with 5% CO₂ for 4 h with 0.1 wt. % HD particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling, the scale bars indicates 10 μm and the brightfield channel is shown.

FIG. 15C is a confocal microscopy image of RAW 264.7 cells in a glass bottom 96-well plate (Cellvis) after being incubated at 37° C. in a humidified atmosphere with 5% CO₂ for 4 h with 0.1 wt. % HD particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling, the scale bars indicates 10 μm and the FITC channel is shown.

FIG. 15D is a confocal microscopy image of RAW 264.7 cells in a glass bottom 96-well plate (Cellvis) after being incubated at 37° C. in a humidified atmosphere with 5% CO₂ for 4 h with 0.1 wt. % HD particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling, the scale bars indicates 10 μm and the TRITC channel is shown.

FIG. 16A is a confocal microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling. Scale bars indicate 10 μm. The merged brightfield, FITC, and 4′,6-diamidino-2-phenylindole (DAPI) channels are shown.

FIG. 16B is a confocal microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling. Scale bars indicate 10 μm. The brightfield channel is shown.

FIG. 16C is a confocal microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling. Scale bars indicate 10 μm. The FITC channel is shown.

FIG. 16D is a confocal microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free DMEM supplemented with 1% penicillin-streptomycin. The cells were washed twice with PBS, fixed by 4% v/v paraformaldehyde in sterile water and stored in PBS, and imaged on confocal with 60× oil immersion objective and Nyquist XY sampling. Scale bars indicate 10 μm. The DAPI channel is shown.

FIG. 17 is a confocal microscopy image of RAW 264.7 cells in a glass bottom 96-well plate (Cellvis) after being incubated at 37° C. in a humidified atmosphere with 5% CO₂ for 4 h with 0.1 wt. % HD-OVA-FITC particle suspension prepared in serum-free DMEM supplemented with 1% penicillin-streptomycin. The cells were washed twice with PBS, fixed by 4% v/v paraformaldehyde in sterile water and stored in PBS. Imaged on confocal with 60× oil immersion objective and Nyquist XY sampling. Scale bars indicating 10 μm. Merged FITC and DAPI channels are shown.

FIG. 18 is a bar graph showing the viability results from a MTS cytotoxicity assay with RAW 264.7 cell line reported as mean±standard deviation. ‘Control’ results were obtained from RAW 264.7 cells in serum-free DMEM supplemented with 1% penicillin-streptomycin. ‘HD-OVA-FITC 0.1 wt. %’, ‘HD 0.1 wt. %’, and ‘DMSO 10% v/v’ results were obtained from RAW 264.7 cells in 0.1 wt. % HD-OVA-FITC particle suspension prepared in serum-free DMEM, 0.1 wt. % HD particle suspension prepared in serum-free DMEM, and 10% v/v DMSO in serum-free DMEM (all supplemented in 1% penicillin-streptomycin), respectively.

### DETAILED DESCRIPTION

The present disclosure relates to carrier particles made of polycations that are “charge-shifting”. These charge-shifting polycations are materials that contain an initial high cationic charge density that allows for a complexation with material (e.g. vaccine, protein, and/or nucleic acid) associated with the carrier particles. The carrier particles can then be subject to cellular uptake where a charge-shifting mechanism will occur, often by hydrolysis, thereby reducing the overall cationic charge density to improve dissociation and release of the complexed material (i.e. the cargo or payload of the polycation carrier particle).

Traditionally, charge-shifting polycations for cellular delivery have been focused on the use of soluble polymers either in branched or linear polymer architectures. A limitation of soluble polycations is that the polyplex formation with nucleic acids is a difficult process to control particle morphology, which can strongly influence particle uptake.

In vivo drug delivery by polymer particles requires eventual degradation into cyto-compatible by-products under physiological conditions. For crosslinked materials, this requires crosslinkers that are degradable. Typically, crosslinkers that contain disulfides, acetals, and ketals have been used as biodegradable functional groups that upon degradation convert the crosslinked polymer particles into soluble polymer. While these functional groups are capable of biodegradation, there are limiting issues associated with them. Acetals and ketals are functional groups that are capable of undergoing acid catalyzed hydrolysis and require basic pH in aqueous solution to ensure material stability before in vivo application. When a carrier particle with acetals and ketals functional groups is endocytosed, the acidic pH of endosomes and lysosomes can trigger the release of the contents of the particle before reaching the cytoplasm or cell nucleus. Accordingly, ketals and acetals may be less desirable functional groups for cellular delivery. Moreover, acetals and ketals crosslinkers are typically relatively hydrophobic, so they are prone to issues with water solubility and protein biniding through hydrophobic interactions. Disulfides are another example of functional groups traditionally used in carrier particles. When the disulfides are cleaved or degraded, they produce free thiol groups which are reactive and can potentially be involved in undesirable side reactions (e.g. with proteins, DNA, etc.). Therefore, disulfides may also be less desirable as functional groups in carrier particles.

The present disclosure provides polycation carrier particles that overcome the above identified disadvantages while providing the advantages of an improved biocompatibility, a reduced cytotoxicity and a biodegradability of the polycation carrier particle as well as the resulting degraded by-products. The polycation carrier particles of the present disclosure can degrade by hydrolysis to form acrylic acid units on polymer, which are non-toxic and commonly used in biocompatible materials. The resulting degraded product is hydrolyzed small molecules, which are biocompatible and can be cleared out of the body over time through renal clearance. In some embodiments, the hydrolyzed small molecules are physiologically inert and are preferably devoid of acrylic groups. The hydrolyzed small molecules can have a chemical structure that generally resembles known food additives, for example dimethyl ethanol. To achieve these improvements, the polycation carrier particles of the present disclosure are formed from an acrylate monomer and one or more hydrophilic co-monomers. The crosslinkers of the present disclosure forming the polycation carrier particle combine the improved performance of charge-shifting materials for polycation based drug delivery with the biodegradability of the crosslinkers. In one embodiment, the polycation carrier particles can be synthesized with a self-stabilizing precipitation polymerization that leverages a balance of polymer solvency and crosslinker loading to yield narrow disperse charge-shifting polycation carrier particles.

The term “acrylate” as used herein refers to a compound having one or more acrylate groups. In some embodiments, the acrylate monomer is a “multiacrylate” monomer having two or more acrylate groups. For example, the compound can be a diacrylate (two acrylate groups), a triacrylate (three acrylate groups) or a tetracrylate (four acrylate groups).

The term “polycation” as used herein, refers to a polymer that has a net positive charge and that is formed by acrylate monomers and one or more hydrophilic co-monomers as described herein.

The term “degradable” as used herein means that a degradation occurs under physiological conditions, with timeframes on the order of 2 hours to 2 weeks, and preferably between 8 hour and 48 hours. The degradation can include the hydrolysis of acrylate and acrylic acid groups and the dissociation of the polycation polymer into small molecules.

The term “optionally substituted” as used herein may be defined as having one or more substitutions selected from a halogen (e.g. Cl, Br, F, and I), an oxide, an amine, an amide, a carboxyl, an alkoxy, a hydroxyl, an alcohol, an ester, an ether, a nitro, a nitrile, an alkyl (e.g. C₁-C₆), an aryl (e.g. phenyl), an acrylamide, and other known substituents that are biocompatible. In some embodiments, the substituents can exclude phosphates, sulfates and other groups that counteract the cationic charge.

In some embodiments, the acrylate monomer of the present disclosure is of formula I

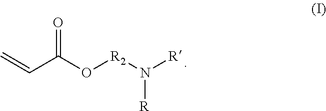

R is a hydrogen, a C₁-C₁₄ linear or branched alkyl, a C₃-C₈ cycloalkyl, or a 5 to 10 membered aryl or heteroaryl ring. R′ is a C₂-C₁₄ linear or branched alkyl, a C₃-C₈ cycloalkyl, or a 5 to 10 membered aryl or heteroaryl ring that is terminated by at least one polymerizable group, preferably an acrylate group, capable of crosslinking with the one or more hydrophilic monomers. The alkyl, cycloalkyl, aryl and heteroaryl can be optionally substituted and optionally interrupted by one or more oxygen, sulfur or nitrogen atoms. The polymerizable group can be selected from an acrylate group, a methacrylate group, a (meth)acrylamide group, or a styrene group. In some embodiments, R and R′ can link and form a heterocycloalkyl containing the nitrogen bound to R and R′ shown in formula I. For example a C₃-C₈ heterocycloalkyl can be formed which is optionally substituted.

In some embodiments, R is defined as hydrogen, a C₁-C₁₃, a C₁-C₁₂, a C₁-C₁₁, a C₁-C₁₀, a C₁-C₉, a C₁-C₈, a C₁-C₇, a C₁-C₆, a C₁-C₅, a C₁-C₄, a C₁-C₃, a C₁-C₂ or a methyl. R can be linear or branched and optionally substituted and optionally interrupted by one or more oxygen, sulfur or nitrogen atoms and optionally terminated with an acrylate group. In some embodiments, R′ is C₂-C₁₄, C₂-C₁₃, C₂-C₁₂, C₂-C₁₁, C₂-C₁₀, C₂-C₉, C₂-C₈, C₂-C₇, C₂-C₆, C₂-C₅, C₂-C₄, C₂-C₃ or is C₂, and is optionally substituted and optionally interrupted by one or more oxygen, sulfur or nitrogen atoms and is terminated by at least one polymerizable group.

The acrylate monomer of the present disclosure is a crosslinker meaning that it has at least two polymerizable groups including the acrylate group linked to R₂ in the formulas shown herein. A monomer having a single polymerizable group would not allow the formation of a polymer particle and would not allow the crosslinking necessary to form such a particle, instead resulting in a linear polymer. In preferred embodiments shown below, the acrylate monomer is a multiacrylate monomer having multiple acrylate groups allowing for improved crosslinking, as having more groups that can react increases the crosslinking efficiency.

R₂ is C₂ or C₃, i.e. —CH₂—CH₂—, —CH₂—CH₂—CH₂— or cyclopropyl, preferably —CH₂—CH₂— or —CH₂—CH₂—CH₂—. In some embodiments, R₂ is not substituted. The acrylate monomer of the present disclosure has an acrylate group neighbouring an amine as shown in formula I with the spacer between the two groups being R₂. This feature of the acrylate monomers allows the polymerization of the acrylate monomer into the polycation carrier particle by reaction of the acrylate group enabled by the amine.

The amine exerts a direct inductive and nucleophilic effect on activating the ester towards hydrolysis. It also helps attract water and hydroxide ions which react with esters during the hydrolysis, and may further activate the ester carbonyl through hydrogen bonding.

In some embodiments, the acrylate monomer is a multiacrylate monomer of the of formula II

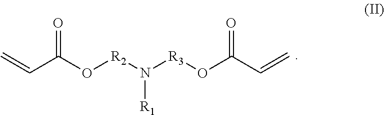

R₁ is hydrogen or a C₁-C₁₄ linear, branched or cyclic alkyl that is optionally substituted and optionally interrupted by one or more oxygen, sulfur or nitrogen atoms and optionally terminated with an acrylate group. R₂ is C₂ or C₃ with C₃ being linear or cyclic, preferably linear. R₃ is a C₂-C₁₄ linear or branched alkyl, that is optionally substituted and optionally interrupted by one or more oxygen, sulfur or nitrogen atoms.

In some embodiments, R₁ is defined as hydrogen, a C₁-C₁₃, a C₁-C₁₂, a C₁-C₁₁, a C₁-C₁₀, a C₁-C₉, a C₁-C₈, a C₁-C₇, a C₁-C₆, a C₁-C₅, a C₁-C₄, a C₁-C₃, a C₁-C₂ or a methyl. R₁ can be linear or branched and optionally substituted and optionally interrupted by one or more oxygen, sulfur or nitrogen atoms and optionally terminated with an acrylate group. In some embodiments, R₃ is C₂-C₁₄, C₂-C₁₃, C₂-C₁₂, C₂-C₁₁, C₂-C₁₀, C₂-C₉, C₂-C₈, C₂-C₇, C₂-C₆, C₂-C₅, C₂-C₄, C₂-C₃ or is C₂, and is optionally substituted and optionally interrupted by one or more oxygen, sulfur or nitrogen atoms.

In some embodiments, the multiacrylate monomer is of formula III

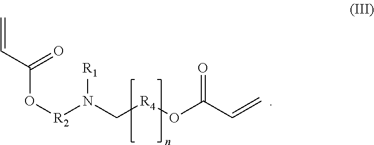

R₁ and R₂ are as previously defined. R₄ is —CH₂—CH₂—O— or —CH₂—CH₂—CH₂—O— and the terminal CH₂ group of R₄ is linked to the oxide of the terminal acrylate group. And, n is an integer selected from 1, 2, 3, or 4.

In some embodiments, the multiacrylate monomer is of formula IV

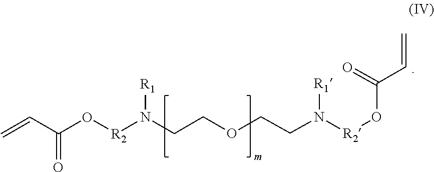

R₁ and R₂ are as previously defined. R₁′ independently follows the same definition provided herein for R₁. R₂′ is C₂-C₃ and m is an integer selected from 0, 1, 2, 3 or 4. R₂′ can be —CH₂—CH₂—, —CH₂—CH₂—CH₂— or cyclopropyl, preferably —CH₂—CH₂— or —CH₂—CH₂—CH₂—. In some embodiments, R₂′ is not substituted.

In some embodiments, the acrylate monomer is of formula V:

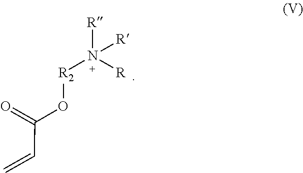

R, R′, and R₂ are as previously defined. R″ independently follows the same definition as R. In some embodiments, two or more of R, R′ and R″ can link and form a heterocycloalkyl containing the nitrogen bound to R, R′ and R″ shown in formula V. For example a C₃-C₈ heterocycloalkyl can be formed which is optionally substituted.

In some embodiments, the acrylate monomer of formula V can be a multiacrylate monomer. In some embodiments, the multiacrylate monomer is of formula VI.

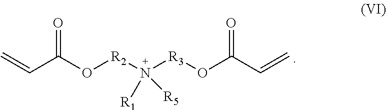

R₁, R₂, and R₃ are as previously defined. R₅ independently follows the definition of R₁ provided above.

In some embodiments, the multiacrylate monomer is of formula VII

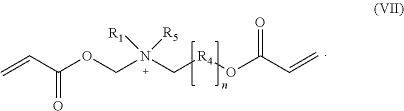

wherein R₁, R₄, R₅, and n are as previously defined.

In some embodiments, the multiacrylate monomer of formula IV can have one or both of the nitrogen atoms being protonated and linked to groups R₅ and R₅′ both independently following the definition of R₁ previously provided. For example, the multiacrylate monomer of formula IV can have both nitrogen protonated as shown below in formula VIII.

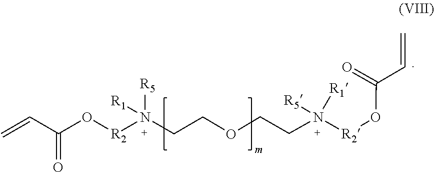

R₁, R₁′, R₂, R₂′, R₅, R₅′, and m are as previously defined.

In preferred embodiments, the acrylate monomer is selected from:

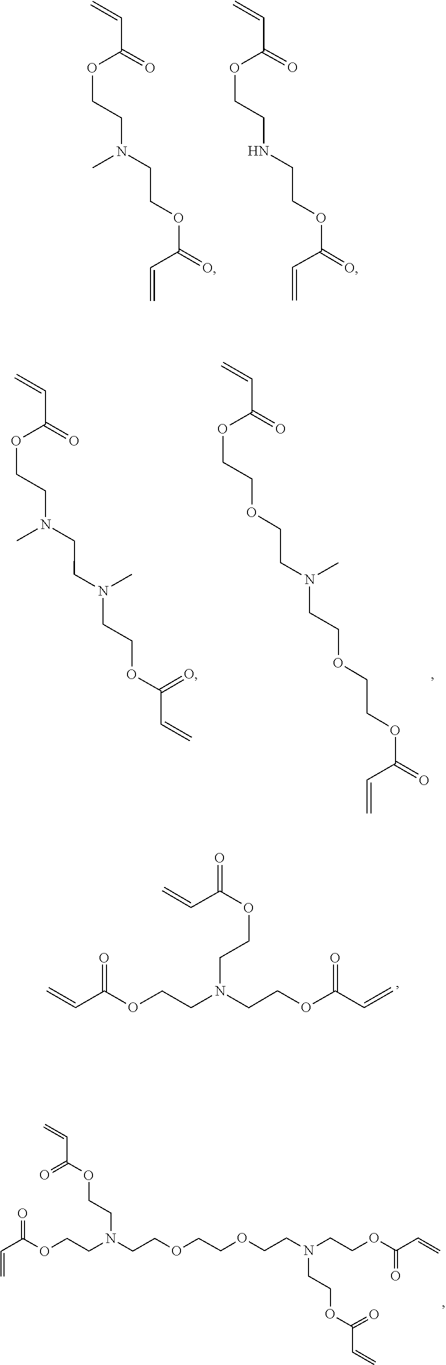 [SMILES: C=CC(=O)OCCN(C)CCOC(=O)C=C ; C=CC(=O)OCCNCCOC(=O)C=C ; C=CC(=O)OCCN(C)CCN(C)CCOC(=O)C=C ; C=CC(=O)OCCOCCN(C)CCOCCOC(=O)C=C ; C=CC(=O)OCCN(CCOC(=O)C=C)CCOC(=O)C=C ; C=CC(=O)OCCN(CCOCCOCCN(CCOC(=O)C=C)CCOC(=O)C=C)CCOC(=O)C=C]

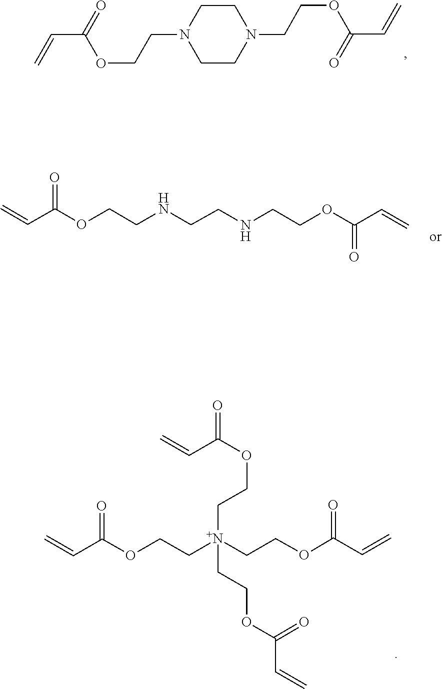

In one embodiment, the acrylate monomer is N-methyldiethanolamine diacrylate, termed (DXL) having the formula:

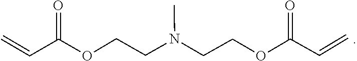 [SMILES: C=CC(=O)OCCN(C)CCOC(=O)C=C]

The acrylate monomer can be a cationic charge shifting monomer that contributes to the net positive charge as well as the charge shifting properties of the resulting polycation. The acrylate monomer is crosslinked or polymerized with one or more hydrophilic monomers to obtain the polycation carrier particles. The acrylate monomer is crosslinked with the one or more hydrophilic monomer to reduce crosslinking density of the resulting polycation. There are many techniques to perform the polymerization of the present monomers including precipitation polymerization, micro emulsion polymerization, dispersion polymerization, inverse-suspension polymerization and inverse emulsion polymerization (i.e. water in oil).

In some embodiments, the hydrophilic monomer is a neutral polar monomer. In some embodiments, the hydrophilic monomer has a molecular weight of less than 500 g/mol, less than 400 g/mol or less than 300 g/mol. A smaller molecule is advantageous in that the hydrophilic monomer becomes more soluble in aqueous phases. In one example, the hydrophilic monomer can be a monoacrylate monomer, monomethacrylate monomer, an acrylamide monomer (e.g. monoacrylamide monomer) or a methacrylamide monomer (e.g. monomethacrylamide monomer). In some embodiments, the hydrophilic monomer can have a net positive cationic charge and accordingly contribute to the overall charge of the polycation carrier particle. By using different hydrophilic monomers and different concentrations of the hydrophilic monomer the overall net charge of the polycation carrier particle can be tuned, which in turn has a role in the release kinetics and binding properties of the polycation carrier particle.

In some embodiments, the hydrophilic monomer is of formula IX

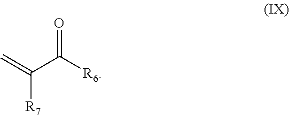

R₆ is —O—(CH₂)ₚ—Y, —NJK or —NJ₂K⁺. p is an integer selected from 2, 3, or 4. Y is defined as OH, COOH, NJ₂, NJ₃⁺, or ONJ₂ and each J is independently defined as H, methyl or ethyl, and K is ethyl, propyl or butyl (optionally branched and optionally substituted). R₇ is hydrogen or methyl.

In some embodiments, the hydrophilic monomer is a monoacrylate monomer of formula X

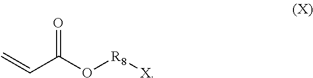

R₈ is a C₂-C₄ linear alkyl and X is selected from hydroxyl, carboxyl, tertiary amine, quaternary ammonium or amide. The amine may be substituted by methyl or ethyl groups.

In some embodiments, the acrylate monomer is crosslinked with one or more hydrophilic monomers in a molar percent ratio of from 15/85 to 50/50, from 20/80 to 40/60, or from 25/75 to 35/65. In some embodiments, only one hydrophilic monomer is used and the acrylate monomer to hydrophilic monomer molar percent ratio is about 30/70. In one embodiment, two hydrophilic monomers are used and the acrylate monomer to hydrophilic monomer ratio is about 33/33/33. The term “about” as used herein can be defined as ±15, ±10, ±9, ±8, ±7, ±6, ±5, ±4, ±3, ±2 or ±1 and the “±” is applied to each value defined in the ratio or on the total value of the ratio.

In some embodiments, the synthesis of a charge-shifting polycation carrier particle is performed with the diacrylate crosslinker DXL. The synthesis can be a precipitation polymerization to produce the degradable, polycation carrier particles. DXL is an example of a diacrylate crosslinker that can be used to form degradable polymer particles due to its hydrolyzable, labile ester groups. Analogous acrylate crosslinkers (e.g. according to formulas I-VIII) that incorporate labile esters with neighbouring amino groups can also be used to prepare degradable particles and polymer materials. As shown in the above formulas I-VIII, examples of DXL derivatives include varying spacer lengths that contain tertiary or secondary amino groups, and tri-, tetra-acrylate analogues. The loading percentage of DXL may be used to form particles of varying size and with various rates of degradation. Copolymerizing DXL with analogues of DXL may also be used to tune the overall rate of degradation of the particles.

The formation of polymer particles of a controlled size may allow the formation of polyplex particles (cationic particle plus payload) of controlled size. Heterogenous polymerization systems to yield polymer particles include emulsion, dispersion, suspension and precipitation polymerizations. Typically, these heterogenous polymerizations require use of surfactants or steric stabilizer for particle formation. These surface stabilizers can leave undesirable chemical modalities on the particle surface groups that may interfere with potential delivery applications.

Precipitation polymerization is an exemplary method to obtain the polycation carrier particles at laboratory scale, but other methods are also contemplated for industrial scale and the like. Precipitation polymerization is a high yield method that can produce mono-disperse, swellable, stabilizer-free particles suitable for use in biomedical applications. Precipitation polymerization is well suited to making particles containing reactive monomer(s), in particular water-sensitive ones, as well as producing narrow-disperse, micron-sized particles that are free of stabilizers or surfactants. In one embodiment, the yield is defined as the weight or molar ratio of starting monomers and optionally initiators to monomers present in the polymer formed. In another embodiment, the yield is defined as the weight or molar ratio of starting monomers and optionally initiators to monomers present in the particles. In various embodiments, the yield for precipitation polymerization can be of at least 1%, at least 2%, at least 3%, at least 5%, and preferably at least 10% or at least 20%. The polycation carrier particles can be obtained by precipitation polymerization of the acrylate monomer with one or more hydrophilic monomers, under particle-forming conditions followed optionally by hydrolysis and/or functionalization. Pendant acrylate groups of the acrylate monomer and/or the hydrophilic monomer can be functionalized with small molecule thiol or amino compounds.

Precipitation polymerization begins with a homogeneous solution of monomers (the acrylate monomer and one or more hydrophilic monomer), and an initiator. A typical total monomer loading is between about 1 to about 20 wt. %, or between about 2 to about 10 wt. % of the solution, and a solvent with the right solvency properties for the polymer that is formed. The formation of particles becomes inefficient and limited with a total monomer loading lower than 1 wt. %. In one embodiment, one or more additional monomers (i.e. not the acrylate monomer nor the hydrophilic monomer as defined herein) may be added to the monomer loading to produce a polymer geared towards a specific application.

The solvent of the precipitation polymerization should be a poor enough solvent to cause the polymer to aggregate and form particles, but still a good enough solvent that the polymer chains on the particle surface are swollen, which prevents particle-particle aggregation during polymerization. In one embodiment, solvents used have Hildebrand solubility parameters about 4 to about 5 MPa½ above or below (i.e., more or less polar) than that of the forming polymer. Furthermore, in some embodiments the viscosity of the solvent is a further factor to consider in the selection of the solvent. A low viscosity solvent is preferred. In one embodiment, the solvent has a viscosity of less than about 0.5 cP at 20° C. The solvent used for precipitation polymerization should have a boiling point greater than the polymerization temperature (typically 60-70° C. for thermally initiated polymerization), and it should not substantially react with the monomers or initiator. In the case of the acrylate monomer, nucleophilic solvents like water, alcohols or amines should be avoided. Particles may also be obtained from photoinitiated precipitation polymerization, which allows lower boiling point solvents to be used. Examples of solvents suitable for the precipitation polymerization of the present disclosure include but are not limited to heptane, toluene, xylenes, methyl ethyl ketone (MEK), tetrahydrofuran (THF), acetonitrile, ethyl acetate, benzene, cyclohexane, chloroform, or mixtures thereof. In the case of photoinitiated polymerization, solvents such as acetone, diethyl ether, dichloromethane and pentane may be used.

Alternative methods to precipitation polymerization to obtain the polycation carrier particles include inverse emulsion polymerization, microemulsion polymerization, inverse-suspension polymerization and dispersion polymerization. Suspension and inverse suspension polymer particles have homogeneous particle properties, as they are formed in essentially mini-bulk polymerizations. Suspension and inverse suspension polymerization carried out using mechanical dispersal of the liquid particle forming phase (e.g., monomer mixture) in a bulk continuous phase usually produce particles with broad size distributions, given the statistical balance of droplet sharing and coalescence found in these processes. Inverse suspension polymerizations of droplets of aqueous solutions of hydrophilic monomers mechanically dispersed in an immiscible, oil-like media can be used to form spherical microparticles and microgels at large scale, though with broad size distributions. Similarly, dispersal of aqueous coacervate phases in a continuous aqueous phase, followed by crosslinking of the dispersed droplets into hydrogel beads, can be seen as an example of aqueous-aqueous suspension polymerization leading to spherical, crosslinked hydrogel particles, though with large particle size distributions. Emulsion type polymerizations use particle initiation in the continuous media, and can result in the large-scale production of narrow-disperse nanoparticles. In both suspension and emulsion type polymerizations, water is typically used as the solvent, which may not be amenable to reactive, hydrolytically unstable monomers. Dispersion polymerization starts with a solution of monomers, initiators and colloidal stabilizers in solvents that are poor for the forming polymer. This process takes advantage of the decreasing solubility of growing polymer chains, and can be used to form mono-disperse microparticles if large amounts of steric stabilizers are used to prevent aggregation of the forming particles.

The polycation carrier particles obtained, can have a size of from 200 nm to 5 μm. This size range is particularly suitable for internalization of the polycation carrier particles in cells (e.g. mammalian cells). The positive charge of the polycation carrier particles also plays a role in promoting cellular uptake. A preferred range for the size of the particles is from 0.5 μm to 5 μm, from 1 μm to 5 μm or from 1 μm to 3 μm. The size can be defined as the diameter when the particles are spheroidal or when the shape is irregular can be defined as the greatest distance between two opposite points taken in a line that goes through the center of the particle. The polycation carrier particles of the present disclosure generally have a spherical shape with a smooth or rough surface. In one embodiment, the shape is a sphere or an irregular sphere. The irregular sphere may be defined as having small bumps on the surface thereby rendering the surface rough.

When the polycation carrier particle is internalized by a cell, it is exposed to specific aqueous or pH conditions that cause the polycation carrier particle to degrade for example through chemical reactions of the reactive esters of the polycation carrier particle. Degradation by hydrolysis of the reactive esters of the acrylate monomer can occur readily at pH 7, but can also occur at other pH. Generally, a faster degradation will occur at a higher pH (e.g. above 7), whereas a slower degradation will occur at a lower pH (e.g. below 7). The composition of polycation carrier particles can be varied by copolymerizing the different acrylate and hydrophilic monomers described above. The hydrophilic monomers can be used to vary the physical and chemical properties of the particles; hydrophilicity, hydrophobicity, charge, rate of degradation.

When the polycation carrier particle degrades, the crosslinks that held the polymer chains together in the particle are broken, and the polymer chains are released into aqueous solution. The polymer chains are comprised of a portion of the now degraded acrylate monomer and small molecules as previously described. The polymer chains produced by particle degradation can be processed and cleared out of a subject's body through renal clearance. Degradation by hydrolysis can occur at physiological conditions (i.e. 37° C., pH 7) and does not require additional harsh external stimuli. Degradation does not occur by free radicals. For that reason, the degradation is considered biocompatible. When DXL is the acrylate monomer, the polymer by-product obtained would be an acrylic acid copolymer, which is a material often used in biomaterial applications, (i.e. dental implants, hydrogels).

The material complexed with the polycation carrier particles can be a vaccine, a genetic material such as polynucleic acids (mRNA, DNA, siRNA), or a therapeutic agent. The polycation carrier particles can be used to deliver the cargo material in an aqueous environment where degradation may occur, such as inside cells. The polycation carrier particles can be used to bind proteins such as ovalbumin and other proteins such as growth factors. Negatively charged cargo material can readily associate with the positively charged particle and is preferred (e.g. nucleic acids). The size of the particles, the charge of the particles and the composition of the particles (monomer selection) can be varied so as to be optimized to a specific delivery route (e.g. intravenous or oral) and for delivery and dissociate of the particles in a specific environment (e.g. pH range).

In some embodiments, the polycation carrier particle associates or encapsulates a vaccine composition and can therefore serve as delivery platform for antigens. Accordingly, the polycation carrier particles may:
Act as carriers for antigens, e.g., RNA including m-RNA, DNA, proteins, viral shell fragments, whole inactivated viral shells, or active innocuous viruses such as adenoviruses that have been modified to express the desired antigen protein;
Have cationic groups that can electrostatically bind the antigen during storage and administration to the recipient's immune system;
Have adjuvant properties to ensure recognition and processing by the host immune system, including where said adjuvant properties are based on cationic or polycationic groups, or on certain carbohydrate groups;
Have compositions where cationic or polycationic groups can be cleaved by spontaneous or enzyme-mediated hydrolysis in order to release the bound antigen payload over a timeframe beneficial to evoking a strong immune response in the recipient;
Have crosslinkers that may undergo slow spontaneous or enzyme-mediated hydrolysis in order to ensure ultimate clearance of the microparticles from the recipient through processes including renal clearance;
Have compositions including cationic and polycations groups as well as non-stoichiometric polyampholytes that can bind antigen for storage at room temperature defined as up to 40° C., and without need for cold-chain logistics during storage and transportation; and
May additionally be loaded with silver nanoparticles to enhance cellular immune response, either by coprecipitation during precipitation polymerization, or by reductive precipitation from silver salts as part of the post-functionalization, or by adsorption of preformed silver nanoparticles onto the described polymer nanoparticles.

Accordingly, in some embodiments, there is provided a vaccine composition or a therapeutic composition comprising the polycation carrier particle of the present disclosure. The polycation carrier particle of the present disclosure can be administered to a subject in need thereof without a further carrier. For example, the polycation carrier particles can be suspended in a solution for intravenous injection. Other injection routes, particularly for vaccine applications, include intramuscular, intranasal and percutaneous (microarray needles) administration. In other embodiments, the polycation carrier particles may be formulated as part of an oral composition (e.g. an oral capsule).

In some embodiments, the polycation carrier particle is complexed with a cargo or payload that elicits an immune response and is administered to a subject. The cargo of the polycation carrier particle can be a nucleic acid, such as DNA and RNA. Accordingly, in other embodiments, there is provided a method of delivering nucleic acids to cells by administering the polycation carrier particle of the present disclosure having a nucleic acid payload. Thus, the polycation carrier particle can be used in gene editing applications such as gene knockdown, and gene editing with clustered regularly interspaced short palindromic repeats (CRISPR), and the like. In some embodiments, there is provided a method of performing gene therapy on a subject in need thereof comprising administering to the subject the polycation carrier particle of the present disclosure with a nucleic acid payload, for example in a plasmid form.

The polycation carrier particles of the present disclosure can also be used in antibacterial materials such as antibacterial surfaces and coatings, and also in waste water management as flocculants.

### EXAMPLE

### Materials

N-Methyldiethanolamine (≥99%), triethylamine (≥99%), acryloyl chloride (≥97%), 2-hydroxyethyl acrylate (HEA, 96%), N,N-(dimethylamino)ethyl acrylate (DMAEA, 98%), methyl ethyl ketone (MEK, ≥99.0%), heptane (99%), 1,4-dioxane (≥99%), acetone (≥99.5%), sterile-filtered water, deuterium oxide (D₂O, 99.9% D), and deuterated chloroform (CDCl₃, 99.9% D), were purchased from Sigma Aldrich. Azobisisobutyronitrile (AIBN, 99.8%) was purchased from DuPont. Dulbecco's modified Eagle medium (DMEM) (high glucose, sodium pyruvate), fetal bovine serum (FBS) (heat inactivated, Canadian origin), NucBlue Live Cell Stain ReadyProbes reagent (Hoechst33342), penicillin streptomycin (10,000 U/mL), ovalbumin fluorescein conjugate (OVA-FITC), ovalbumin Texas Red™ conjugate (OVA-RED), 1× phosphate buffered saline (PBS), and paraformaldehyde (16% (w/v) in aqueous solution methanol free) were purchased from Thermo Fisher Scientific. CellTiter 96 AQueous Non-Radioactive Cell Proliferation Assay was purchased from Promega. 96 well glass bottom plates with high performance #1.5 cover glass were purchased from Cellvis. Hydrochloric acid (HCl) aqueous solution (35-37 wt. %), sodium hydroxide (reagent grade), and disodium hydrogen phosphate (298%) were purchased from Caledon Laboratory Chemicals. Sodium chloride (≥99%) and sodium bicarbonate (≥99.7%) were purchased from ACP Chemicals. Sodium dihydrogen orthophosphate (assured grade) was purchased from BDH Chemicals. Ethyl alcohol (95% vol.) was purchased Commercial Alcohols.

### Synthesis of Methyldiethanolamine Diacrylate (DXL)

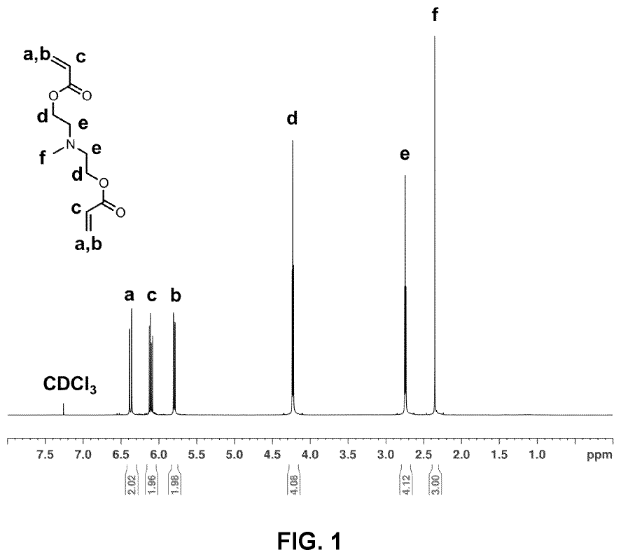

N-Methyldiethanolamine (6 g, 50.4 mmol) and triethylamine (TEA) (12.714 g, 125.9 mmol) were dissolved in 400 mL of dichloromethane (DCM) in a round-bottom flask equipped with a magnetic stir bar and maintained at 0° C. with an external ice-water bath. Acryloyl chloride (11.393 g, 125.9 mmol) was added dropwise over 5-10 min to the stirred reaction mixture, which turned opaque and yellow upon addition. After addition was complete, the reaction was left to stir for overnight, warming up to room temperature. The reaction mixture was washed 4 times with equi-volume fractions of 5 wt. % sodium bicarbonate, followed by brine (saturated sodium chloride solution). The organic layer was dried with anhydrous sodium sulfate, filtered, and concentrated by rotary evaporation to give an orange oil in 90-95% yield. ¹H nuclear magnetic resonance (NMR) was performed and the spectrum is shown in FIG. 1, (CDCl₃, 600 MHz) d 6.37 (2H, dd), 6.10 (2H, dd), 5.80 (2H, dd), 4.23 (4H, t), 2.74 (4H, t), 2.35 (3H, s).

### Hydrolysis of DXL

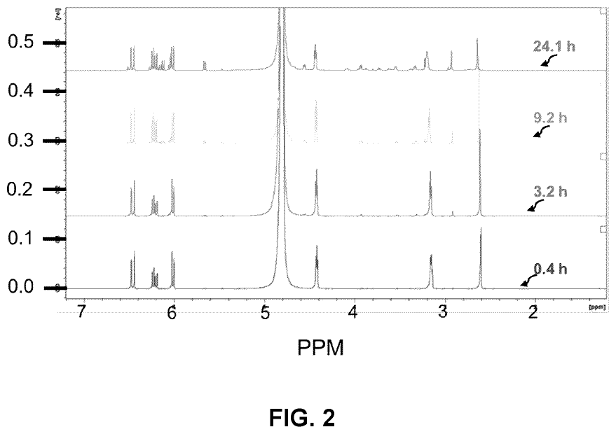

A suspension of DXL was prepared at 0.75 wt. % in 100 mM phosphate buffered D₂O, with pH adjust to pH 7.30 following dissolution of the monomer into the buffer. The solution was transferred to a 5 mm NMR tube and ¹H NMR spectra were recorded with a 500 MHz spectrometer from Bruker at various time intervals while the sample was maintained at room temperature (around 22° C.). The hydrolysis of DXL was measured by calculating the ratio of the alcohol by-products to the diacrylate ester by comparing the integrations of the N-methyl (—CH3) signal of DXL (δ 2.64 ppm), 2-((2-hydroxyethyl)(methyl)amino)ethyl acrylate (δ 2.93 ppm), and N-methyldiethanolamine (δ 2.97 ppm), of the spectra recorded at different times (FIG. 2). Scheme 1 below shows the small-molecule hydrolysis reaction scheme of DXL to form the corresponding alcohol by-products and acrylic acid.

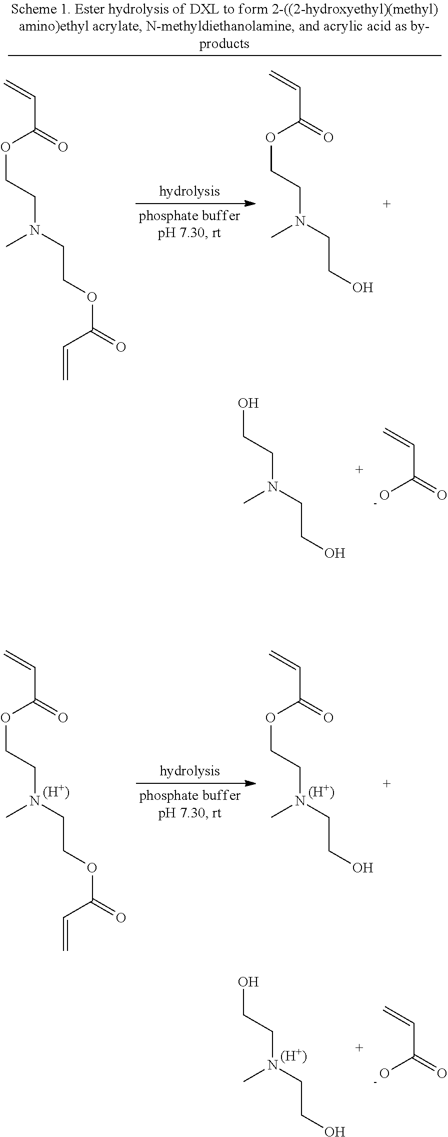

### Synthesis of DXL Crosslinked Particles by Precipitation Polymerization

HEA-DXL (HD) Particles: 2-Hydroxyethyl acrylate (HEA) and methyldiethanolamine diacrylate (DXL) were combined to give mixtures containing 25-30 mol % of DXL. The mixture was dissolved, along with 1 mol % of AIBN as a free-radical initiator for either thermal or photo decomposition, at 5% monomer loading in a solvent mixture of methyl ethyl ketone (MEK) and heptane with relative volume ratios from 70-85% MEK. A sample polymerization mixture targeting 70 mol % HEA and 30 mol % DXL in a 75/25 MEK/heptane solvent mixture at a 1 g total monomer loading is provided as follows: HEA (0.5438 g, 4.684 mmol), DXL (0.4562 g, 2.007 mmol), AIBN (0.0110 g, 0.0669 mmol), MEK (11.4713 g, 14.25 mL), heptane (3.2490 g, 4.75 mL) were combined in a 20 mL glass scintillation vial and placed in a HB-1000 hybridizer oven (UVP) set a 70° C. with rotary mixing at a speed of 7 rpm for a total heating time of 5-8 h. Alternatively, reaction vessels were placed on a set of steel rollers (VIVO electric 12 hotdog and 5 roller grill cooker; model hotdg-v005) set to 3.25 rpm and irradiated with two F15T8/BL fluorescent tube light sources (15 W) at a distance of 1.8 cm from the vials, for a total time of 20-24 h at ambient temperature (22-25° C.).

After polymerization, the reaction mixture was transferred into a 50 mL centrifuge tube, and diluted with an equal volume of 1,4-dioxane. The suspension was then centrifuged at 130 rcf for 15 min, followed by removal of the supernatant and resuspension of the particle pellet in 1,4-dioxane. This washing procedure was repeated for a total of 3 times to remove soluble polymer, residual monomer, and initiator. In the case of samples prepared by thermal polymerization, the particles were sonicated for 20-40 minutes in an effort to separate the particles from larger aggregates. The dried particles were isolated as an orange solid with an isolated yield of 5-10%.

HEA-DMAEA-DXL (HDD) Particles: N,N-(Dimethylamino)ethyl acrylate (DMAEA), DXL, and HEA were combined at relative monomer feed mol ratio of 1:1:1 D:D:H, along with 1 mol % of AIBN as a free-radical initiator for either thermal or photo decomposition, and then dissolved at 5% monomer loading in a solvent mixture of methyl ethyl ketone (MEK) and heptane with relative volume ratios from 70-95% MEK. A sample polymerization mixture targeting a mol ratio of 1:1:1 D:D:H in a 75/25 MEK/heptane solvent mixture at a 1 g total monomer loading is provided as follows: DMAEA (0.2943 g, 2.055 mmol), DXL (0.4671 g, 2.055 mmol), HEA (0.2386 g, 2.055 mmol), AIBN (0.0101 g, 0.0609 mmol), MEK (11.4713 g, 14.25 mL), heptane (3.2490 g, 4.75 mL) were polymerized and isolated as described above for HD particles. The dried HDD particles were obtained as an orange solid with an isolated yield of 1-5%.

### Optical Microscopy Protocol

HEA-DXL (HD) particles were imaged by bright field microscopy using a Nikon Eclipse LV100ND upright microscope, Nikon Ti Eclipse inverted microscope, and a Nikon A1 Confocal Ti Eclipse microscope. The images were analyzed using Nikon NIS-elements Advanced Research software version 5.11.01. Size measurements (n=70) were determined manually using the 3-point circle method.

### Degradation of Particles Protocol

DXL crosslinked particles were prepared at 0.1 wt. % in PBS at pH 7.4 and 0.1 M NaOH (pH ˜11) to monitor particle degradation at physiological conditions and under accelerated conditions, respectively. The suspensions were maintained at room temperature and imaged by brightfield microscopy at various time points.

Loading of Particles with OVA (Ovalbumin) (OVA-FITC (Fluorescein Isocyanate), OVA-RED)

HD particles were sterilized by resuspending them in 70% ethanol at about 0.2 wt. % for 10 min. Following sterilization, particle solutions were prepared and handled using sterile technique in a certified, A2 class 2 biosafety cabinet. The particle suspension in ethanol was then centrifuged at 130 rcf for 15 min, followed by removal of supernatant, then resuspension in sterile water. The washing procedure was repeated for a total of 2 times to remove residual ethanol and form a sterile suspension of particles in PBS at a final concentration of 0.2 wt. %. Separately, a solution of 0.2 wt. % of OVA-FITC was prepared in PBS and sterile filtered using 0.22 mm syringe filter. To load HD particles with OVA-FITC, 400 μL of HD particles at 0.2 wt. % in PBS was transferred to a microcentrifuge tube containing 400 μL of 0.2 wt. % OVA-FITC in PBS. The suspension was mixed by pipetting 10 times followed by 1 min of vortex mixing. The suspension was then centrifuged for 1 min at 3200 rcf, the supernatant was removed, and the pellet was resuspended in 800 μL of PBS. The washing procedure was repeated for a total of 2 times, and the pellet was resuspended in 800 μL of PBS to form a targeted concentration of 0.1 wt. % of HD particles loaded with OVA-FITC (HD-OVA-FITC). The HD-OVA-FITC particles were then imaged by brightfield and fluorescence confocal microscopy to visualize OVA-FITC binding onto the HD particles. HD particles were loaded with OVA-RED in the same manner previously outlined for OVA-FITC.

### Cellular Uptake Protocol

RAW 264.7 macrophage cells (ATCC) were cultured in DMEM supplemented with 10% v/v heat inactivated fetal bovine serum and 1% v/v penicillin-streptomycin (100 units/mL) in T-75 tissue culture treated flasks, maintained at 37° C. in a humidified atmosphere with 5% CO₂. For the cellular uptake assay, RAW 264.7 cells were seeded at a density of 8,000 cells per well with a volume of 100 μL of supplemented media in a glass bottom 96-well plate (Cellvis). The cells were cultured for 3 days to reach about 80% confluence. The media was removed from the wells and the cells were washed once with about 250 μL of PBS. To the washed cells, 100 μL of 0.1 wt. % HD-OVA-FITC particle suspension prepared in serum-free DMEM supplemented with 1% penicillin-streptomycin were added. As controls, cells were exposed to a) 100 μL of serum-free DMEM, b) 100 UL of 10% v/v DMSO in serum-free DMEM, and c) 100 μL of 0.1 wt. % OVA-FITC in serum-free DMEM, were added to the cells. The cells were incubated for 4 h at 37° C., 5% CO₂ in a humidified atmosphere. After incubation, the supernatant was removed from cells and were washed twice with about 250 ml of PBS, followed by addition of 100 μL of 4% v/v paraformaldehyde in sterile water. After 15 min, the paraformaldehyde solution was removed, the cells were washed twice with about 250 μL of PBS, and then 100 μL of PBS was added to the fixed cells. The cells were then imaged by brightfield and confocal fluorescence microscopy

MTS Assay Protocol (i.e. [3-(4,5-dimethylthiazol-2-yl)-5-(3-carboxymethonyphenol)-2-(4-sulfophenyl)-2H-tetrazolium, Salt])

RAW 264.7 macrophage cells (ATCC) were cultured in DMEM supplemented with 10% v/v heat inactivated fetal bovine serum and 1% v/v penicillin-streptomycin (100 units/mL) in T-75 tissue culture treated flasks, maintained at 37° C. in a humidified atmosphere with 5% CO₂. For the cellular uptake assay, RAW 264.7 cells were seeded at a density of 8,000 cells per well with a volume of 100 μL of supplemented media in three tissue culture (TC) treated 96-well plates. The cells were cultured for 3 days to reach around 80% confluence. The media was removed from the wells and the cells were washed once with about 250 μL of PBS. To the washed cells, 100 μL of 0.1 wt. % HD-OVA-FITC particle suspension prepared in serum-free DMEM supplemented with 1% penicillin-streptomycin were added. As controls, 100 μL of serum-free DMEM supplemented with 1% penicillin-streptomycin, 100 μL of 10% v/v DMSO in serum-free DMEM supplemented with 1% penicillin-streptomycin, 100 μL of 0.1 wt. % OVA-FITC in serum-free DMEM supplemented with 1% penicillin-streptomycin, and 100 μL of 0.1 wt. % HD particles suspensions in serum-free DMEM supplemented with 1% penicillin-streptomycin were added to the cells. The cells were incubated for 4 h at 37° C., 5% CO₂ in a humidified atmosphere. 20 μL of MTS solution (Promega) was added to the wells and mixed. The plates were shaken at a horizontal distance of 1 mm for 10 seconds and read at 490 nm using a Biotek™ 800 TS absorbance reader.

### Statistical Analysis

Statistical analysis was performed on the data using one-way analysis of variance (ANOVA) with a Bonferroni post hoc analysis. Significance level was set at p<0.05.

### Hydrolysis of DXL

The hydrolysis of DXL alone was conducted to model the hydrolysis and degradation of DXL crosslinked particles. The degradation of DXL can occur at two reactive ester sites per DXL unit, and it should be noted that hydrolysis of only one of the ester groups is sufficient for degradation of the cross-link. Furthermore, the hydrolysis of the second ester group of the same unit would release N-methyldiethanolamine as a small molecule by-product, while acrylic acid units would remain on the polymer.

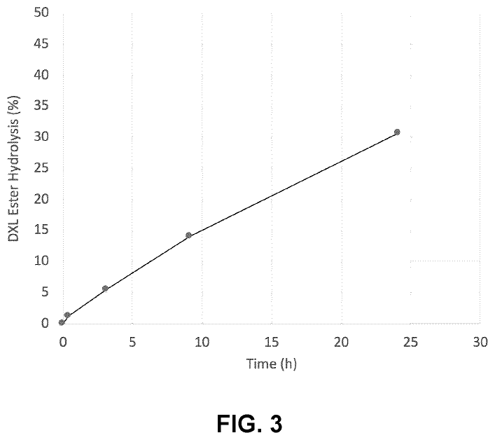

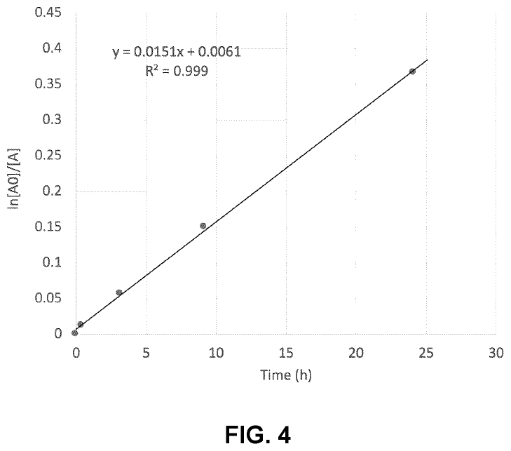

The hydrolysis of DXL in phosphate buffered D₂O at pH 7.3, maintained at room temperature (22° C.), was monitored by ¹H NMR spectroscopy. The results showed that about 31% of DXL was hydrolyzed after 1 day at room temperature (22° C.) (FIG. 3). A small fraction of the monoacrylate product had further hydrolyzed to yield N-methyldiethanolamine (MDEA) such that the product consisted of 27.2% of the monacrylate and 3.4% MDEA. The hydrolysis of one of the ester groups of DXL showed linear pseudo first-order kinetics, with a rate constant (k) of 0.0151 h⁻¹ and a half-life about 1.91 days (FIG. 4).

The results showed that the hydrolysis of DXL occurred about 1.8 times faster than poly[N,N-dimethylaminoethyl acrylate] (PDMAEA) at pH 7, which has a half-life of about 3.5 days. The hydrolysis of DXL polymerized and within the particles is anticipated to occur at a similar rate, with the presence of co-monomers and polarity within the particle have a potential effect on the rate. Neighbouring functional groups incorporated into the structure of DXL or in co-monomers could also be used to tune the rate of hydrolysis and degradation of corresponding particles.

### Precipitation Polymerization of DXL Crosslinked Particles

DXL was used as a diacrylate crosslinker to form degradable, cationic particles in aqueous media. DXL consists of beta amino ester linkages, which have been shown to undergo rapid ester hydrolysis under mild, physiological conditions. N,N-(Dimethylaminoethyl) acrylate (DMAEA) is an example of a monomer with such reactive ester that has been studied in the field of degradable polymer materials. It was hypothesized that DXL crosslinked particles could act as a delivery vehicle for therapeutic payloads such as DNA/RNA and proteins due to its cationic charge with the tertiary ammonium groups. DXL loaded particles could then facilitate cellular uptake with net cationic charge, and then degrade intracellularly with the hydrolysis of the labile ester linkages to release the complexed payload. Degradation reduces the cationic charge on the polymer chain and the soluble polymer can be eliminated from the body by renal filtration.

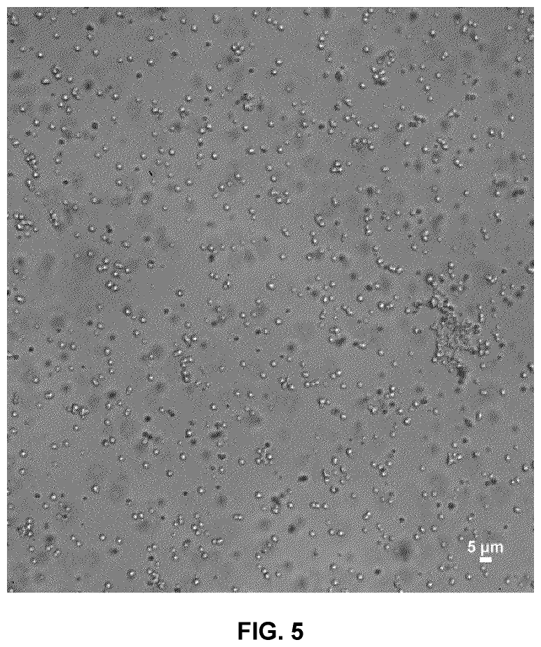

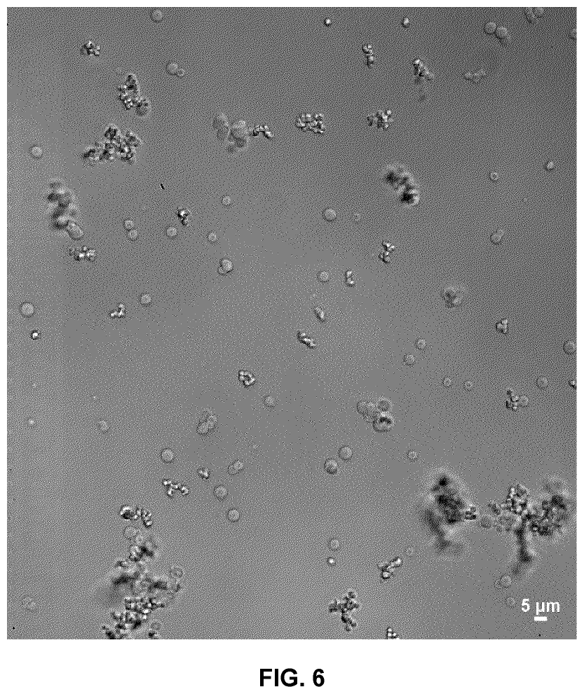

Precipitation polymerization was chosen to prepare DXL crosslinked particles as it does not require the use of surfactants or steric stabilizing agents compared to other methods such as emulsion polymerization. DXL was polymerized with HEA as a neutral, polar co-monomer in a marginal solvent system consisting of MEK as the polar, good solvent and heptane as the non-polar, poor solvent. As HEA-DXL polymer chains form initially in the marginal solvent, aggregation of the polymer chains form particle nuclei that capture subsequent polymer chains that form to grow polymer particles. The HEA/DXL molar feed ratio of 70/30 was chosen due to the limited solubility of DXL in the 75/25 MEK/heptane solvent system that was found to be optimal for particle formation. This formulation was prepared by both photo- and thermal-initiated free-radical polymerization. The size of the particles ranged 2.28±0.35 μm (n=70) for photo-initiated polymerization and 3.60±1.45 μm (n=70) for thermal-initiated polymerization (FIGS. 5 and 6, respectively). It should be noted that the sample prepared by thermal polymerization was sonicated to separate the particles from larger aggregates.

The particles formed through photo-polymerization were smaller and more narrowly dispersed in diameter compared to those formed by thermal-polymerization. The particle formed through photo-polymerization were also more sterically stable.

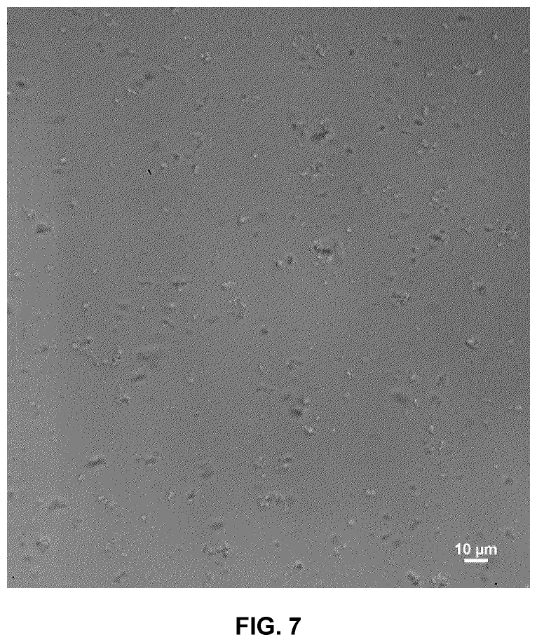

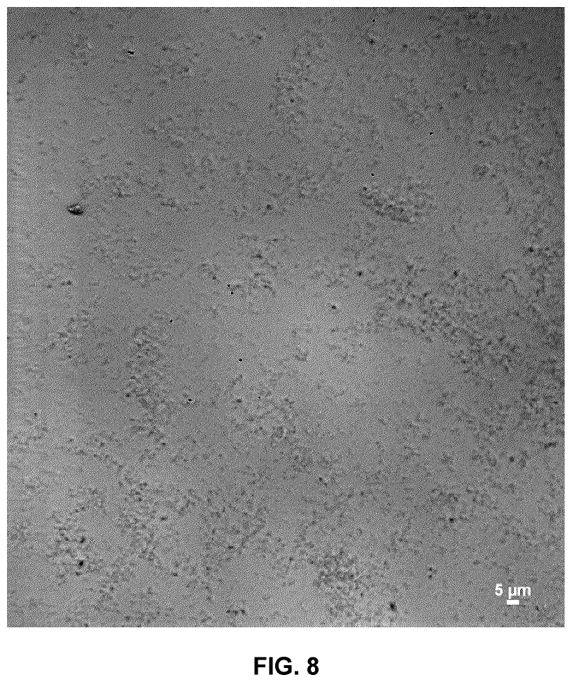

Since DXL showed limited solubility in MEK/heptane solvent mixture compositions suitable for particle formation, DMAEA was copolymerized with DXL and HEA to increase tertiary amine content and cationic charge of the particles. This HEA/DMAEA/DXL terpolymer particle system, herein termed HDD, was attempted in a 1/1/1 molar feed ratio with an 85/15 MEK/heptane solvent mixture and polymerized by thermal- and photo-polymerization. The HDD particles obtained by thermal- and photo-polymerization are shown in FIGS. 7 and 8, respectively.

HDD particles prepared by thermal polymerization resulted in a mixture particles and particle aggregates with a diameter of 1.85±0.47 μm (n=70). HDD particles of the same composition prepared by photo-polymerization resulted in sub-micron particles that could not be analyzed by brightfield microscopy.

Similar to HD particles, the size of the HDD particles of the same composition was smaller when prepared by thermal-polymerization compared to photo-polymerization. The difference is again attributed to the different rate and extent of polymerization as well as the effect of polymerization temperature on solvency.

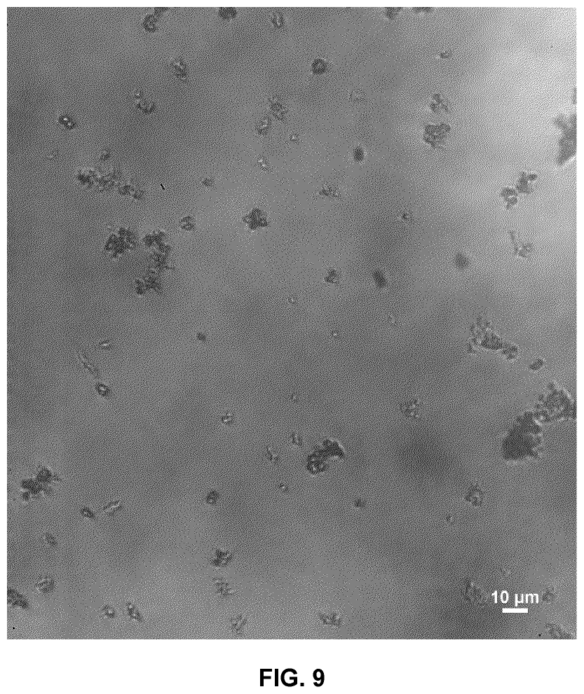

HD particles prepared by photo-polymerization were selected for further proof-of-concept experiments due to the stability of the particle suspension obtained and the relatively narrow size dispersity of the sample. Following purification, HD particles were resuspended in PBS at pH 7.4 which resulted in a mixture of particles and particle aggregates with a diameter of 2.21±0.44 μm (n=70) (FIG. 9).

Loading of HD Particles with OVA (OVA-FITC)

HD particles can bind to proteins, as well as anionic materials such as DNA/RNA, due to the net cationic charge from the tertiary ammonium group of DXL in aqueous media. As HD particles are held together with labile ester linkages of DXL, the particles can degrade and release the bound payload. To demonstrate the ability of the particles to bind proteins for potential drug delivery applications, HD particles were exposed to FITC-labelled ovalbumin (OVA-FITC) or Texas Red-labelled ovalbumin (OVA-RED).

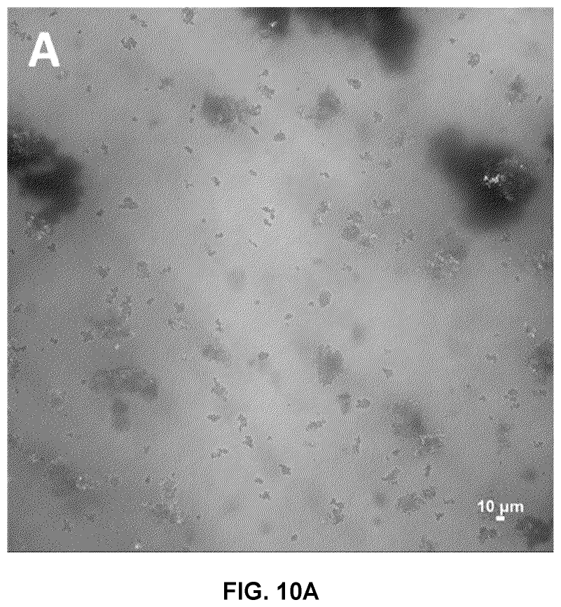

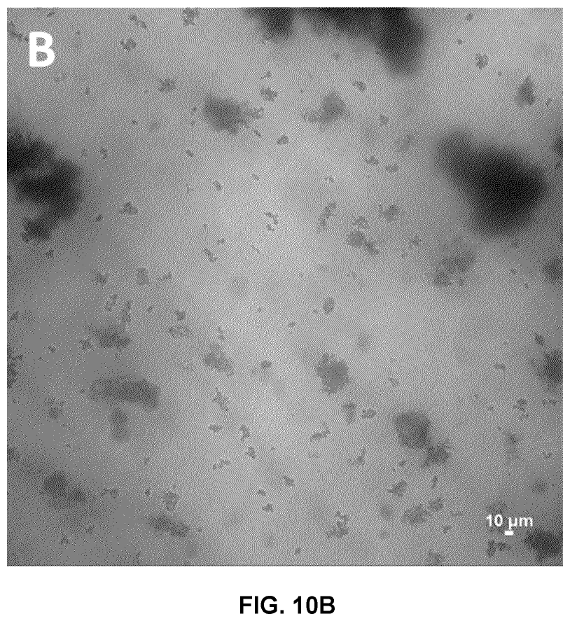

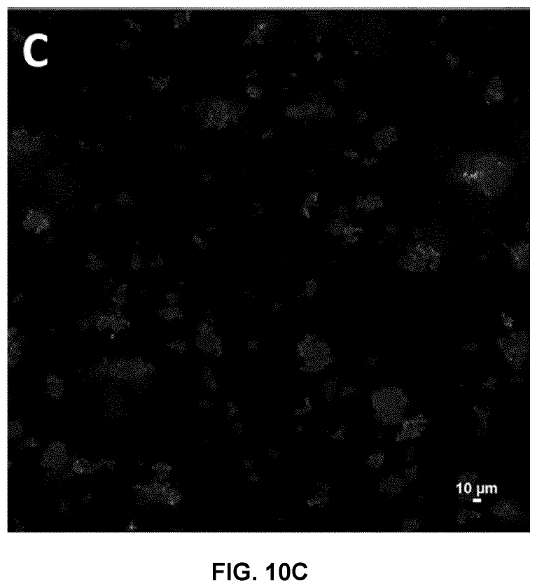

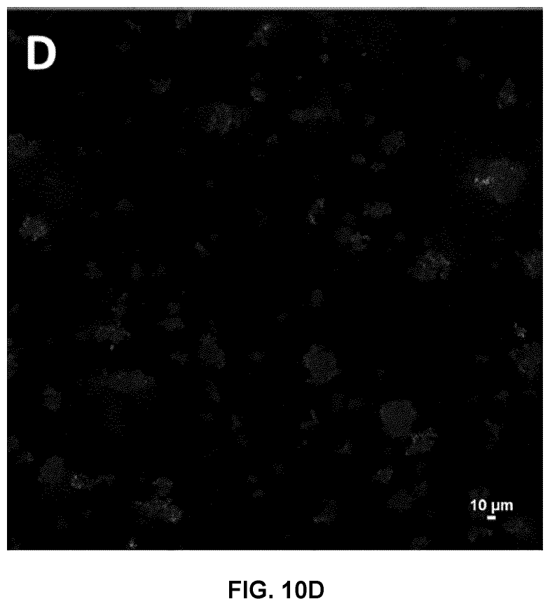

Confocal fluorescence microscopy of unloaded HD particles revealed that there was autofluorescence detected from the particles with greatest intensity in the FITC channel. Fluorescence of the particles may be due to DXL, as tertiary amine-containing polymers have been shown to exhibit fluorescence properties. Fluorescence of HD particles in the TRITC channel was weaker than in the FITC channel under the same acquisition settings (FIGS. 10A-10D).

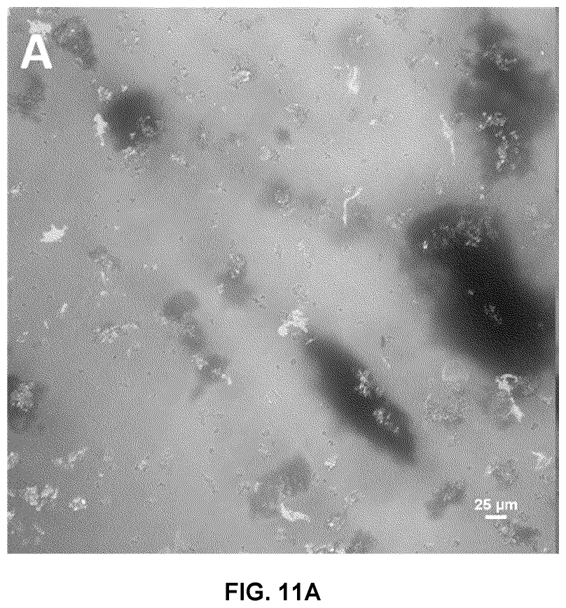

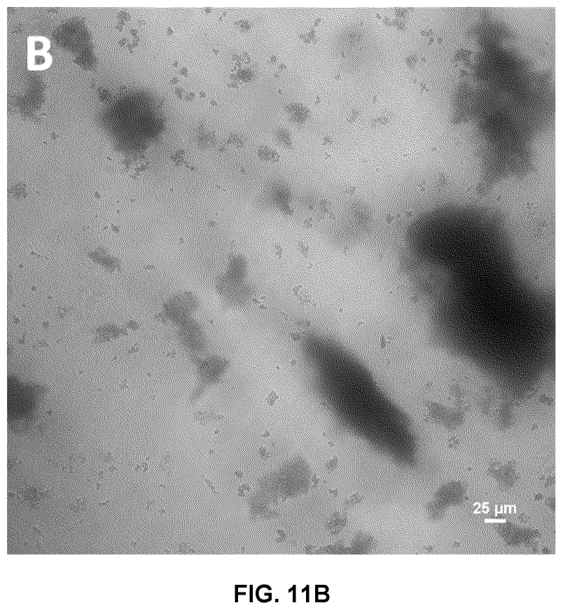

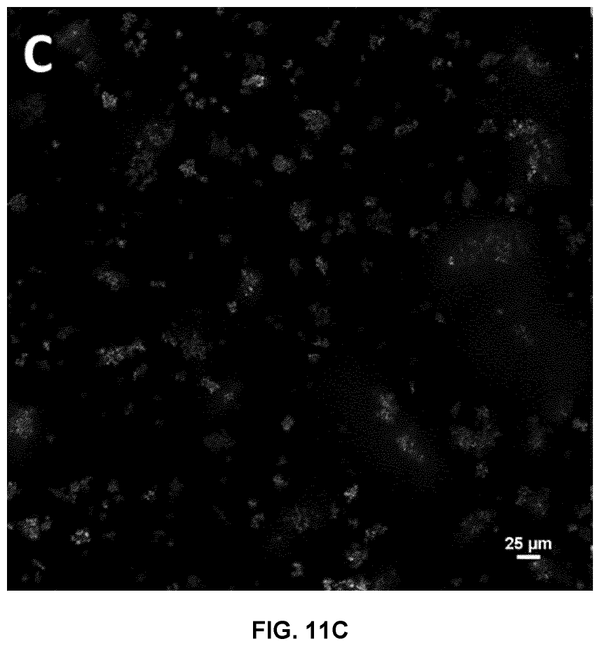

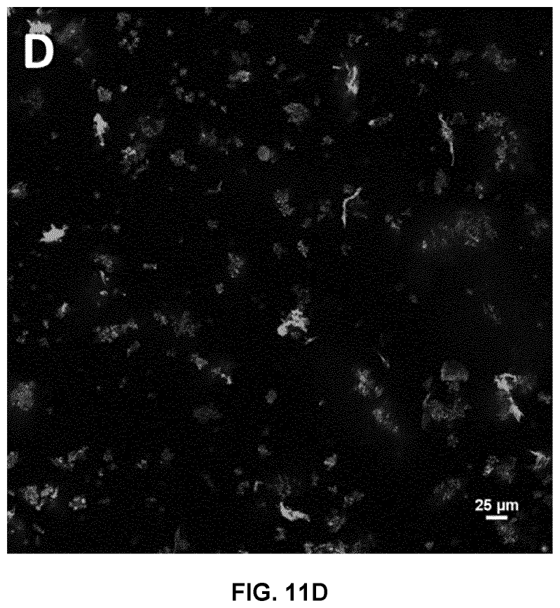

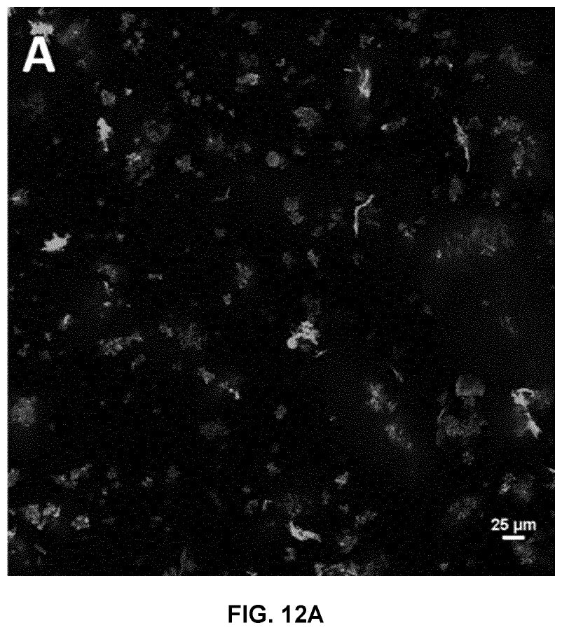

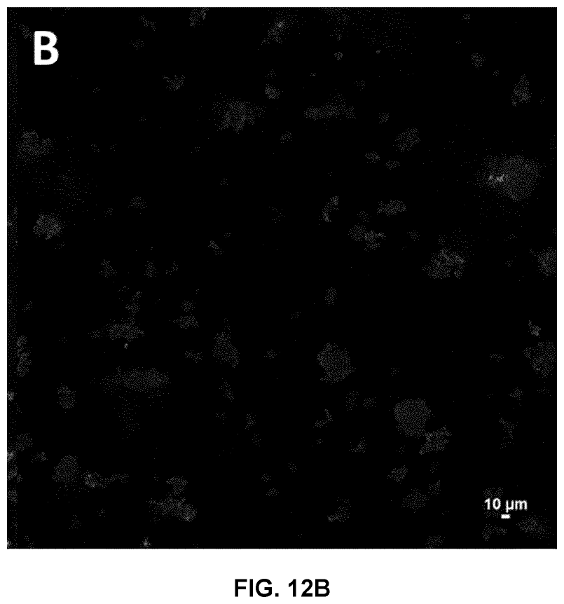

Thus, to minimize interference from HD autofluorescence, OVA-RED was used for binding studies. HD particles loaded with a 1:1 wt ratio of OVA-RED (HD-OVA-RED) were imaged by brightfield and confocal fluorescence microscopy, and the results are shown in FIGS. 11A-11D. When HD particles without OVA-RED were imaged under the same settings, there was much weaker signal in the TRITC channel (FIGS. 12A-12B), suggesting that HD particles are indeed able to bind to proteins such as OVA. It was noted that after binding HD particles with OVA-RED some aggregates of insoluble OVA-RED unbound to HD particles were observed (FIG. 12A). This is likely due to the Texas Red moiety having reduced solubility in aqueous media.

### Cellular Uptake

HD particles loaded with OVA-FITC were exposed to RAW 264.7 macrophage cells as an in vitro model for vaccine delivery. It was hypothesized that HD-OVA particles would be taken up by immune cells and then undergo intracellular degradation by DXL hydrolysis to release the loaded antigen. Other cationic delivery vehicles based on non-charge-shifting polycations have shown promise in binding antigens, however often cause challenges in releasing the antigen. Non-charge-shifting polycations also pose potential risk of damaging cells and cellular components post-delivery due to the toxicity of such polymers.

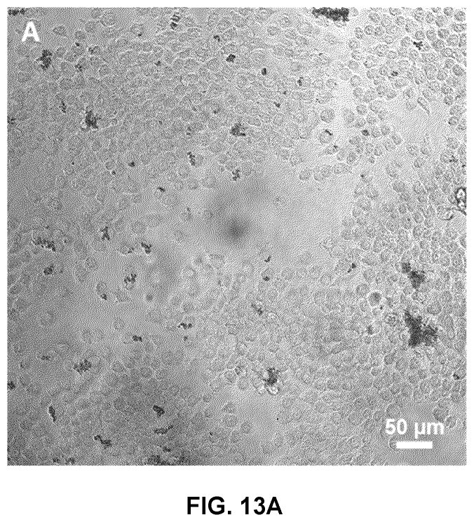

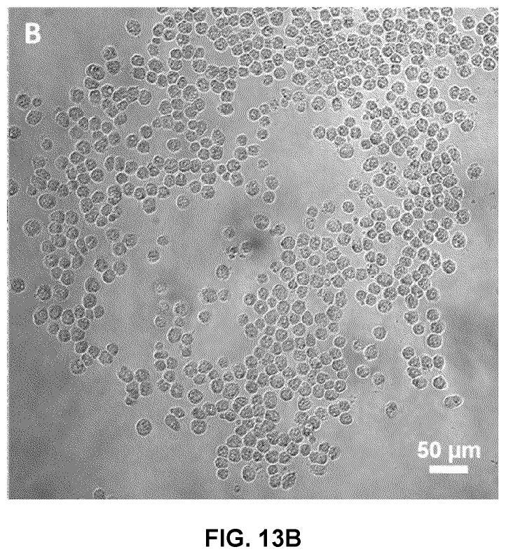

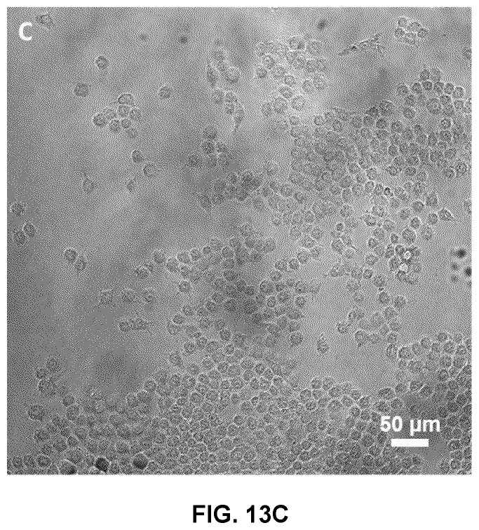

HD-OVA-FITC particles were incubated with RAW 264.7 cells for 4 h, followed by removal of excess particles in the solution by washing twice with PBS. The cells were then imaged by brightfield microscopy (FIGS. 13A-C). FIG. 13A shows the morphology of the cells treated with HD-OVA-FITC particles were similar to untreated cells incubated in DMEM (FIG. 13C). FIG. 13A also shows that a fraction of the HD-OVA-FITC particles appeared to have aggregated to form larger clusters. As a negative control, cells were incubated with 10% v/v DMSO in DMEM, and showed rounded cell morphology and signs of apoptosis (FIG. 13B).

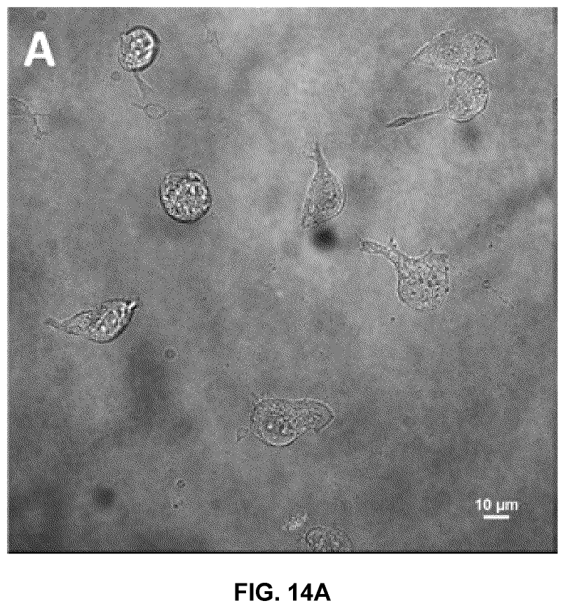

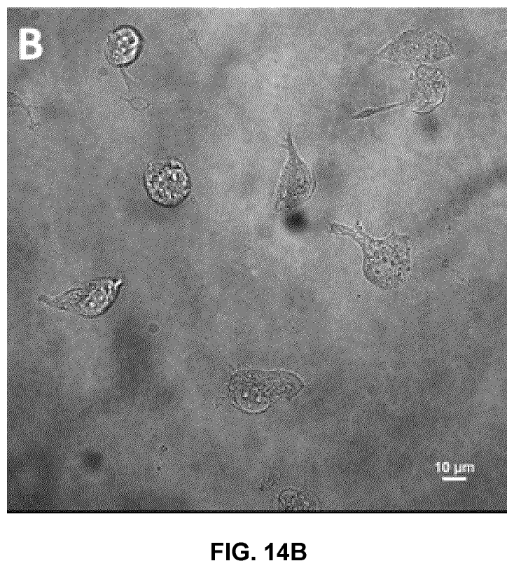

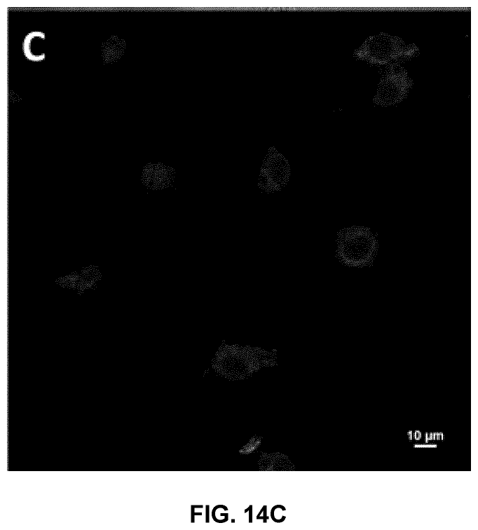

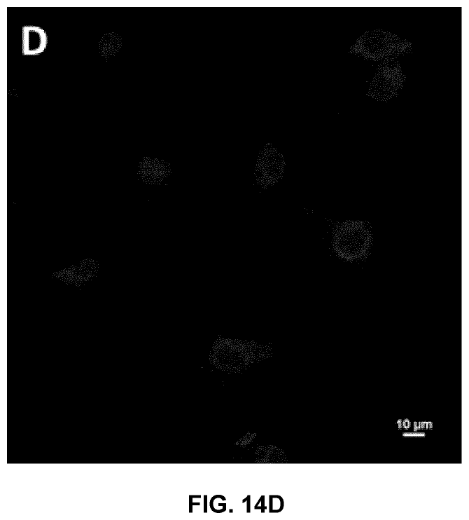

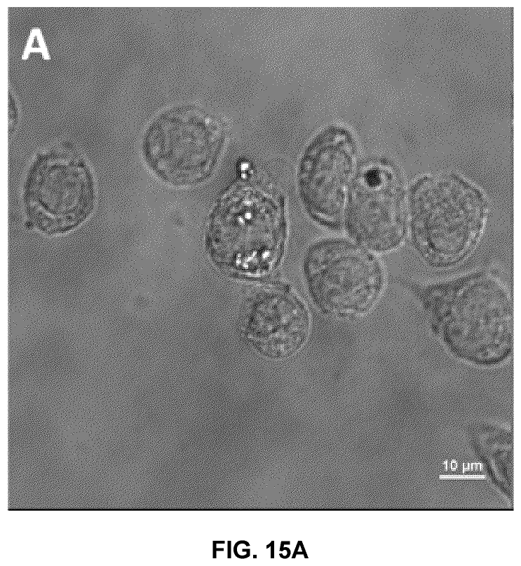

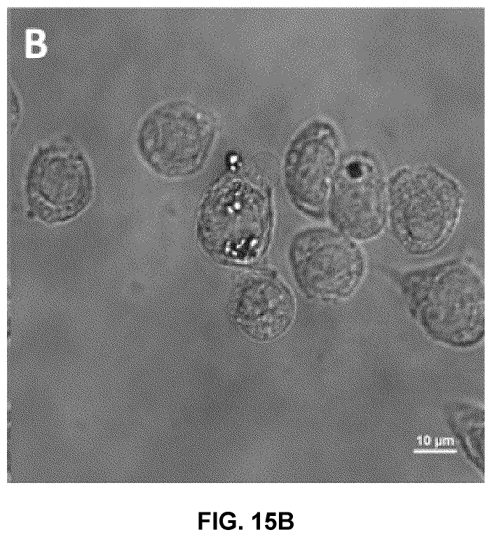

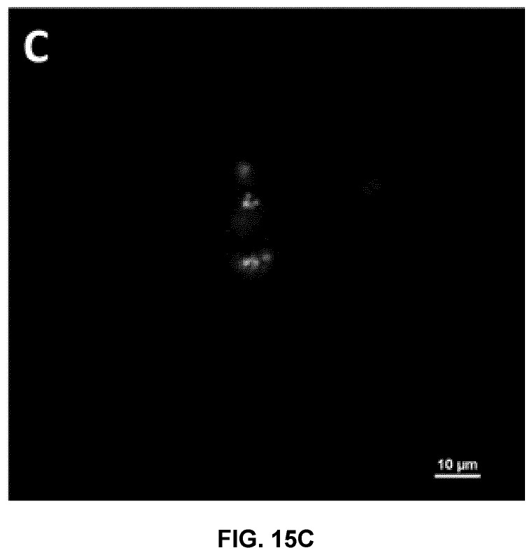

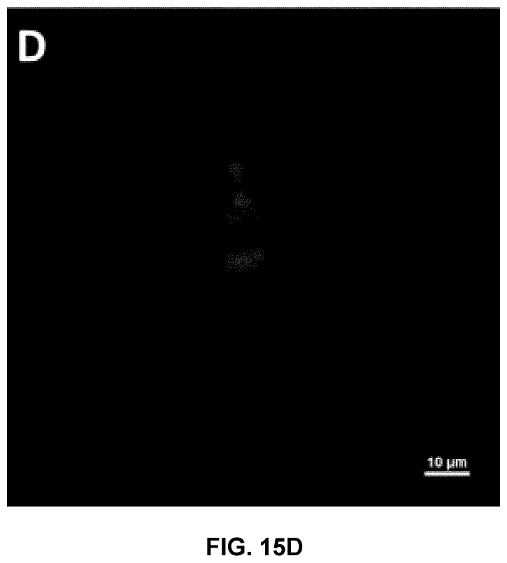

As autofluorescence from both the HD particles and cells may contribute to the signal in the FITC channel, images of the cells alone (FIGS. 14A-14D), and the cells treated with unloaded HD particles (FIGS. 15A-15D) were collected. In neither case was appreciable fluorescence seen from the cells, except for some particle aggregates on the surface of a single cell in FIG. 15.

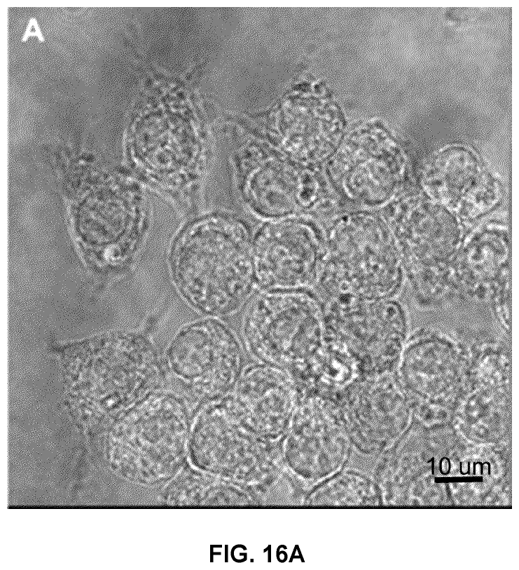

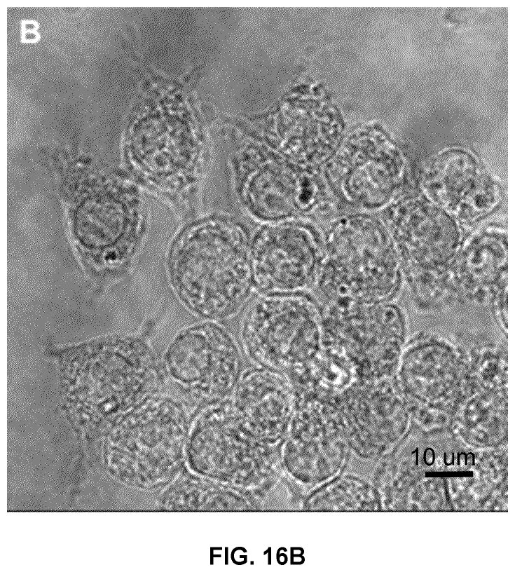

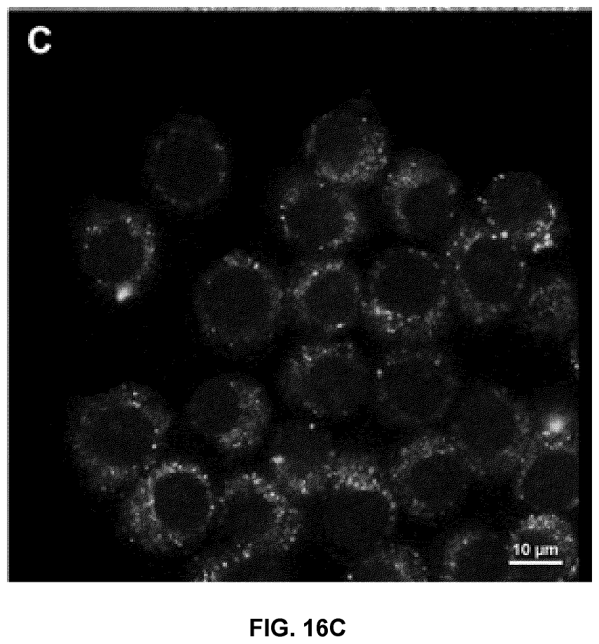

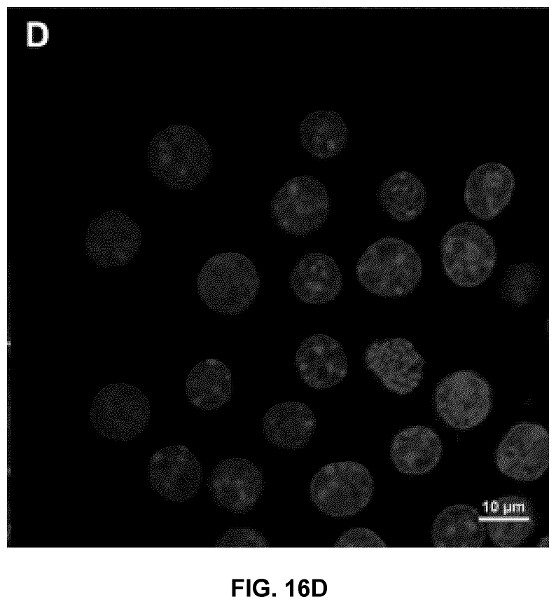

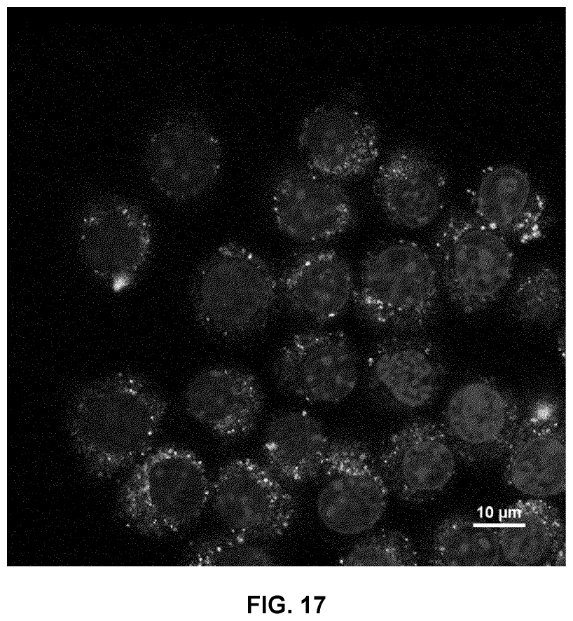

Next the cells were imaged by confocal fluorescence microscopy to check for internalization of the HD-OVA-FITC particles. Images of cells treated with HD-OVA-FITC and then stained with NucBlue nuclear stain are shown in FIGS. 16A-16D. Fluorescence in the FITC channel was observed from the cells, both as diffuse and punctated regions in the cells, suggesting that OVA-FITC had been internalized. Colocalization of the HD-OVA-FITC particles around the cell nuclei was observed through confocal microscopy of merged FITC and DAPI channels (FIG. 17). This suggested that the OVA-FITC had successfully entered the macrophages. These results demonstrate that macrophages will preferentially uptake cationic particles due to their interactions with the negatively charged cell membrane.

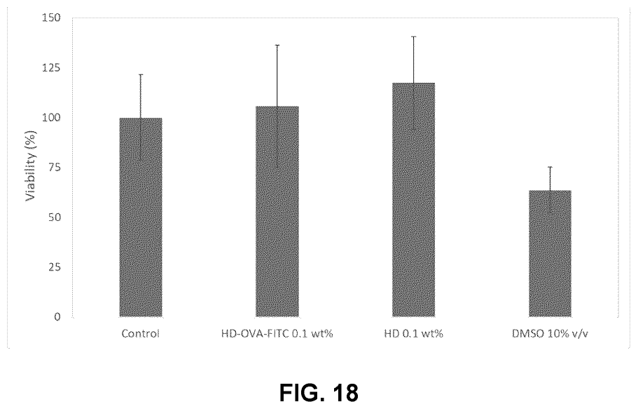

HD-OVA-FITC particles were evaluated for their cytotoxicity with RAW 264.7 cells by using the MTS proliferation assay. As controls, the cells were incubated with serum-free DMEM, 0.1 wt. % HD particle suspension prepared in serum-free DMEM, and 10% v/v DMSO in serum-free DMEM (all supplemented with 1% penicillin-streptomycin). After 4 hours of incubation the mean viability of RAW 264.7 cells were 106±31%, 100±22%, 117±23%, 64±12% respectively (FIG. 18).

The reduced viability of the RAW 264.7 cells in 10% v/v DMSO compared to either sample containing particles (0.1 wt. % HD-OVA-FITC, 0.1 wt. % HD) was statistically significant (p<0.05), whereas the difference in viability of the RAW 264.7 cells in serum free DMEM compared to either sample containing particles was not statistically significant (p>0.05). The results suggested that the HD particles loaded and unloaded with OVA were not significantly cytotoxic at 0.1 wt. % as viability of the treated cells was comparable to the control cells in serum-free media.

What is claimed is:

1. A polycation carrier particle obtained by the polymerization of an acrylate monomer with one or more hydrophilic monomers to form crosslinks, wherein the polycation carrier particle has a net positive electric charge and wherein the acrylate monomer is selected from formula I or formula V 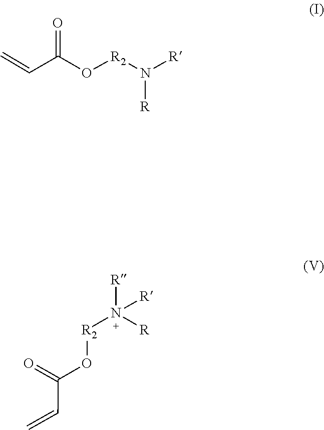
and wherein R and R″ are each independently a hydrogen, a C₁-C₁₄ linear or branched alkyl, a C₃-C₈ cycloalkyl, or a 5 to 10 membered aryl or heteroaryl ring, optionally terminated by a polymerizable group, capable of crosslinking with the one or more hydrophilic monomers,
R′ is a C₂-C₁₄ linear or branched alkyl, a C₃-C₈ cycloalkyl, or a 5 to 10 membered aryl or heteroaryl ring, and R′ is terminated by at least one polymerizable group, capable of crosslinking with the one or more hydrophilic monomers,
the alkyl, cycloalkyl, aryl and heteroaryl can be optionally substituted and optionally interrupted by one or more oxygen, sulfur or nitrogen atoms, and
R₂ is C₂ or C₃ and wherein C₃ is linear or cyclic.

2. The polycation carrier particle of claim 1, wherein the polymerizable group is selected from an acrylate group, a methacrylate group, a (meth)acrylamide group, or a styrene group.

3. The polycation carrier particle of claim 1, wherein the acrylate monomer of formula I is a multiacrylate monomer of formula II 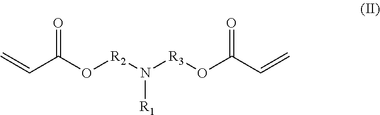
wherein R₁ is hydrogen or a C₁-C₁₄ linear, branched or cyclic alkyl that is optionally substituted and optionally interrupted by one or more oxygen, sulfur or nitrogen atoms and optionally terminated with an acrylate group,
R₂ is C₂ or C₃ and wherein C₃ is linear or cyclic,
R₃ is a C₂-C₁₄ linear or branched alkyl, that is optionally substituted and optionally interrupted by one or more oxygen, sulfur or nitrogen atoms.

4. The polycation carrier particle of claim 3, wherein the multiacrylate monomer is of formula III 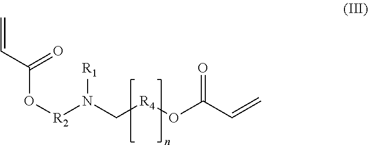
wherein R₁, and R₂ are as defined in claim 1, and R₄ is —CH₂—CH₂—O— or —CH₂—CH₂—CH₂—O— and the terminal CH₂ group of R₄ is linked to the oxide of the terminal acrylate group, and n is an integer selected from 1, 2, 3, or 4.

5. The polycation carrier particle of claim 3, wherein the multiacrylate monomer is of formula IV 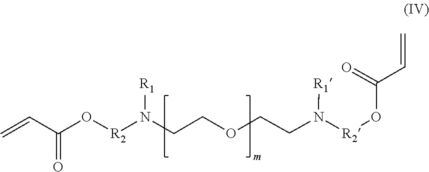
wherein R₁ and R₂ are as defined in claim 1, R₁′ has the same definition as R₁, R₂′ is C₂-C₃ and wherein C₃ is linear or cyclic, and m is an integer selected from 0, 1, 2, 3 or 4.

6. The polycation carrier particle of claim 1, wherein the acrylate monomer of formula V is a multiacrylate monomer of formula VI 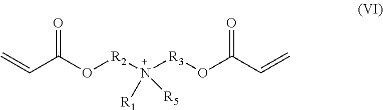
wherein R₁ and R₅ are independently hydrogen or a C₁-C₁₄ linear, branched or cyclic alkyl that is optionally substituted and optionally interrupted by one or more oxygen, sulfur or nitrogen atoms and optionally terminated with an acrylate group,
R₂ is C₂ or C₃ and wherein C₃ is linear or cyclic,
R₃ is a C₂-C₁₄ linear or branched alkyl, that is optionally substituted and optionally interrupted by one or more oxygen, sulfur or nitrogen atoms.

7. The polycation carrier particle of claim 6, wherein the multiacrylate monomer is of formula VII 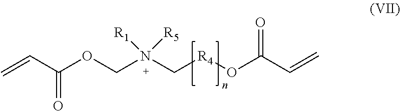
wherein R₁, and R₅ are as defined in claim 6, and R₄ is —CH₂—CH₂—O— or —CH₂—CH₂—CH₂—O— and the terminal CH₂ group of R₄ is linked to the oxide of the terminal acrylate group, and n is an integer selected from 1, 2, 3, or 4.

8. (canceled)

9. The polycation carrier particle of claim 5, wherein the multiacrylate monomer is of formula VIII 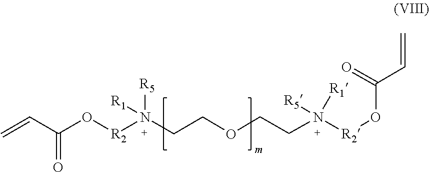
wherein R₁ and R₅ are as defined in claim 6, R₂ is C₂ or C₃ and wherein C₃ is linear or cyclic, m is an integer selected from 0, 1, 2, 3, or 4 and R₁, R₂ and R₅′ independently share the definitions of R₁, R₂ and R₅, respectively.

10. The polycation carrier particle of claim 1, wherein the acrylate monomer is selected from:

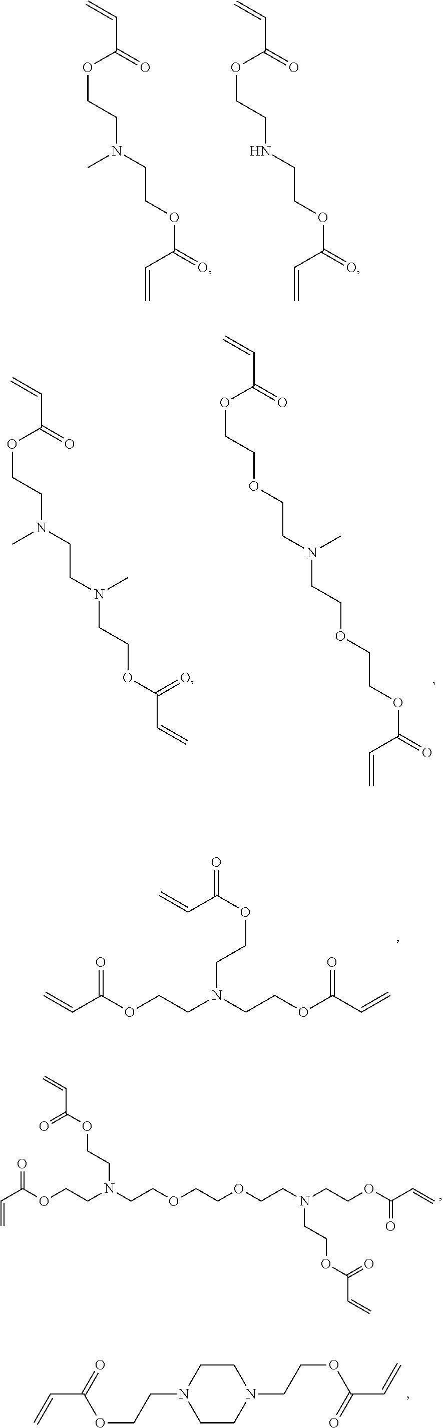

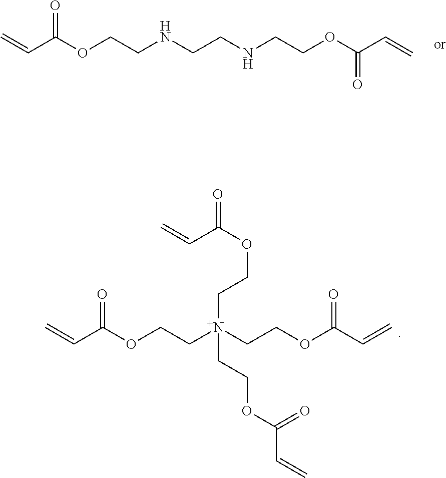

11. The polycation carrier particle of claim 1, wherein the multiacrylate monomer is methyldiethanolamine diacrylate (DXL).

12. The polycation carrier particle of claim 1, wherein the hydrophilic monomer is of formula IX 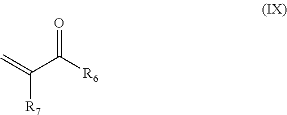
wherein R₆ is —O—(CH₂)ₚ—Y, —NJK or —NJ₂K⁺, p is an integer selected from 2, 3, or 4, Y is defined as OH, COOH, NJ₂, NJ₃⁺, or ONJ₂ and each J is independently defined as H, methyl or ethyl, and K is ethyl, propyl or butyl that is optionally branched and optionally substituted, and R₇ is hydrogen or methyl.

13. The polycation carrier particle of claim 1, wherein the hydrophilic monomer is of formula X 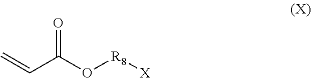
wherein R₈ is a C₂-C₄ linear alkyl and X is selected from hydroxyl, carboxyl, tertiary amine, quaternary ammonium or amide, and the amine is optionally substituted by methyl or ethyl groups.

14. (canceled)

15. The polycation carrier particle of claim 1, wherein the polycation carrier particle has a size of from 200 nm to 5 μm.

16. The polycation carrier particle of claim 1, wherein a molar percent ratio of the acrylate monomer to the one or more hydrophilic monomers is from 15/85 to 50/50.

17. The polycation carrier particle of claim 1, further comprising a cargo material complexed with the polycation carrier particle.

18. (canceled)

19. A method of producing a polycation carrier particle as defined in claim 1, the method comprising:
providing an acrylate monomer as defined in claim 1 and one or more hydrophilic monomers as defined in of claim 1, and,
crosslinking the acrylate monomer with the one or more hydrophilic monomers.

20. The method of claim 19, wherein the step of crosslinking comprises performing one of a precipitation polymerization, a micro emulsion polymerization, a dispersion polymerization, an inverse-suspension polymerization or an inverse emulsion polymerization.

21. The method of claim 19, wherein only one hydrophilic monomer is provided and the acrylate monomer to the hydrophilic monomer molar percent ratio is 30±15/70±15.

22. The method of claim 19, wherein two hydrophilic monomers are provided and the acrylate monomer to the two hydrophilic monomers molar percent ratio is 33±10/33±10/33±10.

23-27. (canceled)

28. A method of inducing an immune response in vivo comprising administering to a subject in need thereof the polycation carrier particle of claim 1 complexed with an antigen or a nucleic acid encoding an antigen or encapsulating a vaccine composition.

29-32. (canceled)
